(19) United States
(12) Patent Application Publication
(10) Pub. No.: US 2025/0002849 A1
(43) Pub. Date: Jan. 2, 2025

(54) TRANSDIFFERENTIATION OF NON-HAIR FOLLICLE STEM CELLS TO NON-AUDITORY INDUCED HAIR FOLLICLE STEM CELLS

(71) Applicant: dNovo Inc., San Jose, CA (US)

(72) Inventor: Ernesto Lujan, San Jose, CA (US)

(21) Appl. No.: 18/688,296

(22) PCT Filed: Sep. 1, 2022
(86) PCT No.: PCT/US2022/075828

§ 371 Date: Feb. 29, 2024

Related U.S. Application Data
(60) Provisional application No. 63/239,474, filed on Sep. 1, 2021.

(51) Int. Cl. C12N 5/071

(52) U.S. Cl. CPC C12N 5/0628; C12N 2501/606; C12N 2506/1307

(57) ABSTRACT

Provided are compositions of a system and methods of use thereof to transdifferentiate non-hair follicle stem (HFS) cells into non-auditory induced hair follicle stem cells (iHFSCs) and to subsequent use of these cells in the treatment of hair loss.

### CROSS-REFERENCE TO RELATED APPLICATIONS

This application claims the benefit of and priority to U.S. Provisional Patent Application No. 63/239,474, filed Sep. 1, 2021, the disclosure of which is hereby incorporated by reference in its entirety for all purposes.

### FIELD OF THE INVENTION

This invention generally relates to compositions for transdifferentiation of non-hair follicle stem (HFS) cells into non-auditory-induced hair follicle stem cells (iHFSCs) for use in treating hair loss.

### BACKGROUND

Effective therapies to treat hair loss (otherwise known as alopecia) have been extensively researched for decades, but currently available treatments are insufficient for long term reversal of the condition. Pharmaceutical therapies can be used to treat underlying conditions that drive hair loss, such as the use of Minoxidil (Rogaine) or Finasteride (Propecia) for hereditary baldness. However, many patients see little, if any, benefit from these therapies, and those patients that do benefit from pharmaceutical intervention must remain on the medication indefinitely to maintain therapeutic effects.

Hair transplantation surgery is an alternative to pharmaceutical intervention. With this procedure, patient hair follicles are taken from a donor site and re-grafted to another part of the scalp as an autograft. Unfortunately, this procedure is only possible in patients exhibiting early stages of hair loss as a sufficient amount of patient donor sites are necessary to perform the procedure. As hereditary hair loss progresses, these donor hair sites become smaller in area and number making successful transplantation less probable. Additionally, hair transplantation surgery may be disappointing as it leads to a decrease in hair density at the donor site, and hair density across the entire scalp will never reach pre-hair loss density due to a lack of donor hair follicles. More recently, laser therapy has been approved for the treatment of hereditary hair loss, however the long-term effects of this therapy are unclear. As a consequence, new therapeutic strategies are needed to promote long-term hair growth that do not require indefinite medication or rely on a finite supply of donor hair follicles.

A promising therapeutic strategy may be the use of stem cells to repopulate cells within hair follicles. Similar to how bone marrow transplantation may be used to reverse hematopoietic disease, hair follicle stem cells (HFSCs) may be used to reverse hair loss. This therapy would provide the benefits of hair transplants without the need to rely on a finite source of patient donor hair follicles by using cells that may self-renew and provide long-term reversal of hair loss.

A major challenge to the use of HFSCs is the ability to obtain an effective amount to use in therapy. HFSCs are low prevalence and must be harvested from existing hair follicles. Furthermore, there are no known methods of transdifferentiating non-hair follicle stem (HFS) cells into induced hair follicle stem cells (iHFSCs). Therefore, any method of efficiently generating iHFSCs from non-HFS cells and effectively grafting them into patient skin would present a promising step in the treatment of hair loss.

### BRIEF SUMMARY OF THE INVENTION

The present disclosure is directed to compositions of a system and methods of use thereof to transdifferentiate non-hair follicle stem (HFS) cells into (iHFSCs) and to subsequent use of these cells in the treatment of hair loss.

Provided herein is a composition for transdifferentiation comprising a population of non-HFS cells: a population of non-auditory iHFSCs expressing one or more marker(s) selected from the group consisting of E-Cadherin (ECAD), keratin 15 (KRT15), Kruppel Like Factor 5 (KLF5), HR Lysine Demethylase and Nuclear Receptor Corepressor (HR), SRY-Box Transcription Factor 9 (SOX9), Transcription Factor AP-2 Alpha (TFAP2A), Integrin Subunit Alpha 6 (ITGA6), BAF Chromatin Remodeling Complex Subunit BCL11B (CTIP2), Epithelial Splicing Regulatory Protein 1 (ESRP1), and T-box Transcription Factor 1 (TBX1); and a hair follicle stem cell reprogramming (HFSCR) system comprising one or more HFSCR factor(s) selected from the group consisting of a p53 agent, a NF-I agent, an Lhx agent, a TFAP2 agent, an IRX agent, and a Myc agent, wherein the HFSCR system causes transdifferentiation of one or more non-HFS cell into one or more non-auditory iHFSC.

In some embodiments, the population of non-auditory iHFSCs have at least a two-fold increased expression of one or more marker(s), selected from the group consisting of ECAD, KRT15, KLF5, HR, SOX9, TFAP2A, ITGA6, CTIP2, ESRP1, and TBX1, as compared to the population of non-HFS cells. In some embodiments, the population of non-auditory iHFSCs have at least a five-fold increased expression of one or more marker(s), selected from the group consisting of ECAD, KRT15, KLF5, HR, SOX9, TFAP2A, ITGA6, CTIP2, ESRP1, and TBX1, as compared to the population of non-HFS cells. In some embodiments, the population of non-auditory iHFSCs have at least a ten-fold increased expression of one or more marker(s), selected from the group consisting of ECAD, KRT15, KLF5, HR, SOX9, TFAP2A, ITGA6, CTIP2, ESRP1, and TBX1, as compared to the population of non-HFS cells. In some embodiments, expression of the one or more marker(s) is maintained over at least one cell passage. In some embodiments, expression of the one or more marker(s) is determined by messenger RNA analysis using a method selected from the group consisting of quantitative polymerase chain reaction (PCR), reverse transcription PCR, RNA-Seq, or northern blot. In some embodiments, expression of the one or more marker(s) is determined by protein analysis using a method selected from the group consisting of flow cytometry, or western blot.

In some embodiments, the p53 agent is a p53 polypeptide, or functional fragment thereof, fused to a permeant domain. In some embodiments, the p53 agent is a nucleic acid encoding a p53 polypeptide, or functional fragment thereof. In some embodiments, the NF-I agent is an NF-I polypeptide, or functional fragment thereof, fused to a permeant domain. In some embodiments, the NF-I agent is a nucleic acid encoding an NF-I polypeptide, or functional fragment thereof. In some embodiments, the Lhx agent is an Lhx polypeptide, or functional fragment thereof, fused to a permeant domain. In some embodiments, the Lhx agent is a nucleic acid encoding a Lhx polypeptide, or functional fragment thereof. In some embodiments, the TFAP2 agent is a TFAP2 polypeptide, or functional fragment thereof, fused to a permeant domain. In some embodiments, the TFAP2 agent is a nucleic acid encoding a TFAP2 polypeptide, or functional fragment thereof. In some embodiments, the IRX agent is an IRX polypeptide, or functional fragment thereof, fused to a permeant domain. In some embodiments, the IRX agent is a nucleic acid encoding an IRX polypeptide, or functional fragment thereof. In some embodiments, the Myc agent is a Myc polypeptide, or functional fragment thereof, fused to a permeant domain. In some embodiments, the Myc agent is a nucleic acid encoding a Myc polypeptide, or functional fragment thereof. In some embodiments, the nucleic acids comprise a tetracycline-responsive promoter. In some embodiments, the nucleic acid is within a capsid of a viral particle. In some embodiments, the permeant domain comprises an amino acid sequence selected from the group consisting of: RQIKIWFQNRRMKWKK (SEQ ID NO:1), RKKRRQRRR (amino acids 49-57 of HIV-1 tat: SEQ ID NO:2), TRQARRNRRRRWRERQR (amino acids 34-50 of HIV-1 rev: SEQ ID NO: 3), RRRRRRRRR (R9; SEQ ID NO:4), and RRRRRRRR (R8: SEQ ID NO:5).

In some embodiments, the population of non-HFS cells are mammalian cells. In some embodiments, the population of non-HFS cells are human or murine cells. In some embodiments, the non-HFS cells are selected from the group consisting of dermal cells, epithelial cells, adipocytes, and hematopoietic cells. In some embodiments the dermal cells are selected from the group consisting of fibroblasts, smooth muscle cells, and connective tissue cells.

Also provided herein is a method of making a population of non-auditory iHFSCs, comprising obtaining a population of non-HFS cells; contacting the population of non-HFS cells with a HFSCR system comprising one or more HFSCR factor(s) selected from the group consisting of a p53 agent, an NF-I agent, an Lhx agent, a TFAP2 agent, an IRX agent, and a Myc agent; and incubating the population of non-HFS cells in the presence of the HFSCR system, wherein one or more non-HFS cell is transdifferentiated into one or more non-auditory iHFSC.

In some embodiments of a method provided herein, the non-HFS cell samples are collected from a mammalian subject, for example, a human or murine subject. In some embodiments, the non-HFS cells are selected from the group consisting of dermal cells, epithelial cells, adipocytes, and hematopoietic cells. In some embodiments the dermal cells are selected from the group consisting of fibroblasts, smooth muscle cells, and connective tissue cells. In some embodiments, the population of non-HFS cells are incubated in the presence of the HFSCR system is for a period of 24 hours to 6 weeks. In some embodiments, the non-HFS cell samples are collected from a subject to whom the transdifferentiated one or more non-auditory iHFSC is administered. In some embodiments, the non-HFS cells are collected from a subject who is different than the subject to whom the transdifferentiated one or more non-auditory iHFSC is administered.

In some embodiments of a method provided herein, the one or more non-auditory iHFSC expresses one or more marker(s) selected from the group consisting of ECAD, KRT15, KLF5, HR, SOX9, TFAP2A, ITGA6, CTIP2, ESRP1, and TBX1. In some embodiments, the population of non-auditory iHFSCs have at least a two-fold increased expression of one or more marker(s) selected from the group consisting of ECAD, KRT15, KLF5, HR, SOX9, TFAP2A, ITGA6, CTIP2, ESRP1, and TBX1, as compared to the population of non-HFS cells. In some embodiments, the population of non-auditory iHFSCs have at least a five-fold increased expression of one or more marker(s) selected from the group consisting of ECAD, KRT15, KLF5, HR, SOX9, TFAP2A, ITGA6, CTIP2, ESRP1, and TBX1, as compared to the population of non-HFS cells. In some embodiments, the population of non-auditory iHFSCs have at least a ten-fold increased expression of one or more marker(s) selected from the group consisting of ECAD, KRT15, KLF5, HR, SOX9, TFAP2A, ITGA6, CTIP2, ESRP1, and TBX1, as compared to the population of non-HFS cells.

In some embodiments, the method comprises passaging the non-auditory iHFSCs at least once. In some embodiments, expression of the one or more marker(s) is maintained over at least one cell passage.

In some embodiments of a method provided herein, the p53 agent is a p53 polypeptide, or functional fragment thereof, fused to a permeant domain. In some embodiments, the p53 agent is a nucleic acid encoding a p53 polypeptide, or functional fragment thereof. In some embodiments, the NF-I agent is an NF-I polypeptide, or functional fragment thereof, fused to a permeant domain. In some embodiments, the NF-I agent is a nucleic acid encoding an NF-I polypeptide, or functional fragment thereof. In some embodiments, the Lhx agent is an Lhx polypeptide, or functional fragment thereof, fused to a permeant domain. In some embodiments, the Lhx agent is a nucleic acid encoding a Lhx polypeptide, or functional fragment thereof. In some embodiments, the TFAP2 agent is a TFAP2 polypeptide, or functional fragment thereof, fused to a permeant domain. In some embodiments, the TFAP2 agent is a nucleic acid encoding a TFAP2 polypeptide, or functional fragment thereof. In some embodiments, the IRX agent is an IRX polypeptide, or functional fragment thereof, fused to a permeant domain. In some embodiments, the IRX agent is a nucleic acid encoding an IRX polypeptide, or functional fragment thereof. In some embodiments, the Myc agent is a Myc polypeptide, or functional fragment thereof, fused to a permeant domain. In some embodiments, the Myc agent is a nucleic acid encoding a Myc polypeptide, or functional fragment thereof. In some embodiments, the nucleic acids comprise a tetracycline-responsive promoter. In some embodiments, the nucleic acid is within a capsid of a viral particle. In some embodiments, the permeant domain comprises an amino acid sequence selected from the group consisting of: RQIKIWFQNRRMKWKK (SEQ ID NO: 1), RKKRRQRRR (amino acids 49-57 of HIV-1 tat: SEQ ID NO:2), TRQARRNRRRRWRERQR (amino acids 34-50 of HIV-1 rev: SEQ ID NO:3), RRRRRRRRR (R9); SEQ ID NO:4), and RRRRRRRR (R8: SEQ ID NO:5).

In some embodiments of a method provided herein, the population of non-auditory iHFSCs are selected and enriched based on the expression of the one or more marker(s) selected from the group consisting of ECAD, KRT15, KLF5, HR, SOX9, TFAP2A, ITGA6, CTIP2, ESRP1, and TBX1. In some embodiments, selecting and enriching comprises fluorescence-activated cell sorting (FACS) or magnetic-activated cell sorting (MACS). In some embodiments, selecting and enriching comprises dissociating the population of non-auditory iHFSCs to form a cell suspension and adding a population of dermal cells to the cell suspension. In some embodiments the dermal cells are selected from the group consisting of fibroblasts, smooth muscle cells, connective tissue cells, dermal papilla cells, and adipocytes.

In some embodiments of a method provided herein, the population of non-auditory iHFSCs are dissociated to form a cell suspension and a population of dermal cells is added to the cell suspension. In some embodiments the dermal cells are selected from the group consisting of fibroblasts, smooth muscle, connective tissue cells, dermal papilla cells, and adipocytes.

In some embodiments of a method provided herein, the non-auditory iHFSCs are cultured on a scaffold. In some embodiments, the scaffold is composed of a plastic, Matrigel or a similar basement membrane extract, gelatin, collagen, or laminin. In some embodiments the non-auditory iHFSCs are cultured on the scaffold for 30 minutes to 48 hours.

In some embodiments of a method provided herein, the non-auditory iHFSCs on the scaffold are grafted onto a subject. In some embodiments, at least 100,000 non-auditory iHFSCs are grafted onto the subject. In some embodiments, at least 1,000,000 non-auditory iHFSCs are grafted onto the subject. In some embodiments, at least 10,000,000 non-auditory iHFSCs are grafted onto the subject. In some embodiments, non-auditory iHFSCs and dermal cells on the scaffold are grafted onto a subject, In some embodiments, at least 100,000 non-auditory iHFSCs and dermal cells are grafted onto the subject. In some embodiments, at least 1,000,000 non-auditory iHFSCs and dermal cells are grafted onto the subject. In some embodiments, at least 10,000,000 non-auditory iHFSCs and dermal cells are grafted onto the subject.

Also provided herein is a method for treating a subject having a hair loss condition, comprising administering a composition comprising a population of non-HFS cells: a population of non-auditory iHFSCs expressing one or more marker(s) selected from the group consisting of ECAD, KRT15, KLF5, HR, SOX9, TFAP2A, ITGA6, CTIP2, ESRP1, and TBX1; and a HFSCR system comprising one or more HFSCR factor(s) selected from the group consisting of a p53 agent, a NF-I agent, an Lhx agent, a TFAP2 agent, an IRX agent, and a Myc agent, wherein the HFSCR system causes transdifferentiation of one or more non-HFS cell into one or more non-auditory iHFSC.

In some embodiments, compositions provided herein are administered to a subject in a method for treating a hair loss condition. For example, in some embodiments, the hair loss condition is selected from the group consisting of androgenic alopecia, alopecia areata, telogen effluvium, trichotillomania, traction alopecia, tinea capitis, cicatricial alopecia, burning or scarring. In some embodiments, the non-auditory iHFSCs are transdifferentiated from non-HFS cells obtained from the subject. In some embodiments, the non-auditory iHFSCs are transdifferentiated from non-HFS cells obtained from a subject different than the subject to which the composition is administered.

Also provided herein is a composition of iHFSCs produced by the process comprising obtaining a population of non-auditory iHFSCs, obtaining a population of non-HFS cells; contacting the population of non-HFS cells with HFSCR system comprising one or more HFSCR factor(s) selected from the group consisting of a p53 agent, an NF-I agent, an Lhx agent, a TFAP2 agent, an IRX agent, and a Myc agent; and incubating the population of non-HFS cells in the presence of the HFSCR system, wherein one or more non-HFS cell is transdifferentiated into one or more non-auditory iHFSC.

### BRIEF DESCRIPTION OF THE DRAWINGS

FIGS. 1A-1J are graphs showing qPCR analysis of canonical hair stem cell markers expressed by human iHFSCs produced by overexpressing TP63, NFIB, LHX2, TFAP2A, IRX4 and MYC in cultured human fibroblasts (HFs). Expression of hair stem cell marker genes E-Cadherein (ECAD; FIG. 1A), keratin 15 (KRT15; FIG. 1B), Kruppel Like Factor 5 (KLF5; FIG. 1C), HR Lysine Demethylase and Nuclear Receptor Corepressor (HR; FIG. 1D), SRY-Box Transcription Factor 9 (SOX9; FIG. 1E), Transcription Factor AP-2 Alpha (TFAP2A; FIG. 1F), Integrin Subunit Alpha 6 (ITGA6; FIG. 1G), BAF Chromatin Remodeling Complex Subunit BCL11B (CTIP2; FIG. 1H), Epithelial Splicing Regulatory Protein 1 (ESRP1; FIG. 1I), and T-box Transcription Factor 1 (TBX1; FIG. 1J), were compared to control HFs.

FIGS. 2A-2O are micrographs showing immunofluorescent staining of human HFSC protein markers. FIGS. 2A, 2D, 2G, 2J. and 2M show 20× phase contrast images, FIGS. 2B, 2E, 2H, 2K, and 2N show DAPI staining for nuclei visualization, and FIGS. 2C, 2F, 2I, 2L, and 2O show staining of ECAD, KRT15, KLF5, SOX9, and ITGA6, respectively, in iHFSCs overexpressing TP63, NFIB, LHX2, TFAP2A, IRX4 and MYC (top micrographs) and negative control HFs (bottom micrographs) stained under the same conditions. Scale bars represent 200 μm.

FIGS. 3A-3E show flow cytometry plots of iHFSCs overexpressing TP63, NFIB, LHX2, TFAP2A, IRX4 and MYC and FIGS. 3F-3J show flow cytometry plots of negative control HFs. FIGS. 3A-3C and FIGS. 3F-3H show forward scatter (FSC) and side-scatter (SSC) gating parameters for cell size. FIG. 3D and FIG. 3I show propidium iodide (PI) staining for exclusion of dead cells. FIG. 3E and FIG. 3J show Keratin 15 and E-cadherin expression. The indicated rectangular gates and percentages indicate the KRT15+/ECAD+ cell population.

FIG. 4 shows graphs of gene expression assessed by qPCR in iHFSCs at passage (P) 2, 5, 7 and 10 following overexpression of TP63, NFIB, LHX2, TFAP2A, IRX4 and MYC, as well as in negative control human fibroblast HFs. Expression values of KRT15 (FIG. 4A) and ECAD (FIG. 4B) were normalized to actin.

FIG. 5 shows images of mouse skin following transplant of iHFSCs. FIGS. 5A and 5B show pictures of iHFSC transplantation and time-matched negative control 4 weeks following transplantation, respectively. FIGS. 5C and 5D show pictures from two independent transplantation experiments in which hair growth was assessed 6 weeks after transplantation (FIG. 5C) and 9 weeks after transplantation (FIG. 5D).

FIGS. 6A-6J are graphs showing qPCR analysis of canonical hair stem cell markers expressed by human iHFSCs produced by overexpressing TP63, NFIB, LHX2, TFAP2A, IRX4 and MYC in cultured human epithelial cells (EPIs). Expression of hair stem cell marker genes ECAD (FIG. 6A), KRT15 (FIG. 6B), KLF5 (FIG. 6C), HR (FIG. 6D), SOX9 (FIG. 6E), TFAP2A (FIG. 6F), ITGA6 (FIG. 6G), CTIP2 (FIG. 6H), ESRP1 (FIG. 6I), and TBX1 (FIG. 6J), were compared to control EPIs.

FIGS. 7A-7J are graphs showing qPCR analysis of canonical hair stem cell markers expressed by human iHFSCs produced by overexpressing TP63, LHX2, TFAP2A, and MYC in cultured human epithelial cells (EPIs). Expression of hair stem cell marker genes ECAD (FIG. 7A), KRT15 (FIG. 7B), KLF5 (FIG. 7C), HR (FIG. 7D), SOX9 (FIG. 7E), TFAP2A (FIG. 7F), ITGA6 (FIG. 7G), CTIP2 (FIG. 7H), ESRP1 (FIG. 7I), and TBX1 (FIG. 7J), were compared to control EPIs.

FIGS. 8A-8J are graphs showing qPCR analysis of canonical hair stem cell markers expressed by human iHFSCs produced by overexpressing TP63, LHX2, TFAP2A, and MYC in cultured human fibroblasts (HFs). Expression of hair stem cell marker genes ECAD (FIG. 8A), KRT15 (FIG. 8B), KLF5 (FIG. 8C), HR (FIG. 8D), SOX9 (FIG. 8E), TFAP2A (FIG. 8F), ITGA6 (FIG. 8G), CTIP2 (FIG. 8H), ESRP1 (FIG. 8I), and TBX1 (FIG. 8J), were compared to control HFs.

FIGS. 9A-9J are graphs showing qPCR analysis of canonical hair stem cell markers expressed by human iHFSCs produced by overexpressing LHX2, TFAP2A, and MYC in cultured human fibroblasts (HFs). Expression of hair stem cell marker genes ECAD (FIG. 9A), KRT15 (FIG. 9B), KLF5 (FIG. 9C), HR (FIG. 9D), SOX9 (FIG. 9E), TFAP2A (FIG. 9F), ITGA6 (FIG. 9G), CTIP2 (FIG. 9H), ESRP1 (FIG. 9I), and TBX1 (FIG. 9J), were compared to control HFs.

### DETAILED DESCRIPTION OF THE INVENTION

The present invention provides compositions and methods of using and producing a population of induced non-auditory hair follicle stem cells (iHFSCs) from a population of non-hair follicle stem (HFS) cells. These methods and compositions find use in producing hair follicle stem cells (HFSCs) and hair follicle progenitor cells (HFPCs) thereof for transplantation; as an experimental model for evaluating therapeutics; and as a source of lineage- and cell-specific products, and the like, for example, for use in treating human hair loss conditions such as androgenic alopecia, alopecia areata, telogen effluvium, trichotillomania, traction alopecia, tinea capitis, and cicatricial alopecia. Also provided are compositions and methods for screening candidate agents for activity in transdifferentiating non-HFS cells into non-auditory iHFSCs. These and other objects, advantages, and features of the invention will become apparent to those persons skilled in the art upon reading the details of the subject compositions and methods as more fully described below.

Unless defined otherwise, all technical and scientific terms used herein have the meaning commonly understood by one of ordinary skill in the art to which this disclosure belongs. To facilitate an understanding of the present invention, a number of terms and phrases are defined below.

As used herein, the singular forms “a,” “an,” and “the” include plural referents unless the context clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm).

A “stem cell” is a cell characterized by the ability of self-renewal through mitotic cell division and the potential to differentiate into the cell types that contribute to a tissue or an organ. Among mammalian stem cells, embryonic and somatic stem cells may be distinguished. The term “embryonic stem cell” is used to refer to the pluripotent stem cells of the inner cell mass of the embryonic blastocyst. These cells are capable of giving rise to an entire organism. The term “somatic stem cell” refers to any pluripotent or multipotent stem cell that differentiates into and maintains fetal, juvenile, and adult tissues. Unlike embryonic stem cells, somatic stem cells cannot give rise to an entire organism.

Pluripotent stem cells, which include embryonic stem cells, embryonic germ cells and induced pluripotent cells, can contribute to tissues of a prenatal, postnatal or adult organism.

The terms “primary cells,” “primary cell lines,” and “primary cultures,” are used interchangeably herein to refer to cells and cell cultures that have been derived from a subject and allowed to grow in vitro for a limited number of passages, i.e. splittings, of the culture.

The terms “treatment,” “treating,” “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it: (b) inhibiting the disease symptom, i.e. arresting its development; or (c) relieving the disease symptom, i.e. causing regression of the disease or symptom.

The terms “individual,” “subject,” “host,” and “patient” are used interchangeably herein and refer to any mammalian subject for whom diagnosis, treatment, or therapy is desired, particularly humans.

### Hair Follicle Stem Cells and Hair Follicle Progenitor Cells

As used herein, the term “hair follicle stem cell” (HFSC) refers to self-renewing, multipotent cells that give rise to hair follicle lineages. By self-renewing, it is meant that when they undergo mitosis, they are capable of producing at least one daughter cell that is a HFSC. By multipotent it is meant that it is capable of giving rise to hair follicle progenitor cells that give rise to cell types of the hair follicle and differentiated hair, i.e. hair shaft cell lineages, inner root sheath cell lineages, and/or outer root sheath cell lineages. HFSCs are capable of contributing to a new hair structure in vivo when using techniques known to those skilled in the art. HFSCs are not pluripotent, that is, they are not capable of giving rise to cells of other non-ectodermal organs.

As used herein, the terms “hair follicle progenitor cell” (HFPC) and “hair follicle progenitor” (HFP) refer to the downstream progeny of HFSCs that arise during development. HFPCs have limited self-renewal capacity. By self-renewal, it is meant that when they undergo mitosis, they produce at least one daughter cell that is a HFPC. By limited self-renewal capacity, it is meant that HFPCs can self-renew for a limited number of mitoses (i.e. 1, 2, 3, 5, or 10 mitoses) before giving rise to two daughter cells that are no longer HFPCs. HFPCs may be multipotent or unipotent. By multipotent it is meant that they are capable of giving rise to HFPCs that give rise to different cell types of the hair follicle and differentiated hair, i.e. hair shaft cell lineages, inner root sheath cell lineages, and/or outer root sheath cell lineages. By unipotent it is meant that HFPCs are capable of giving rise to HFPCs that in turn give rise to single cell types of the hair follicle and differentiated hair, i.e. hair shaft cell lineages, inner root sheath cell lineages, and outer root sheath cell lineages. HFPCs are not pluripotent, meaning they are not capable of giving rise to cells of other non-ectodermal lineages. In addition, HFPCs are mitotic, and so can incorporate BrdU into their DNA or exhibit evidence of cell division as known to those skilled in the art, such as cell division that occurs during cell passaging. HFPCs are capable of contributing to a new hair structure in vivo when using techniques known to those skilled in the art. Due to the fact that HFPCs have similar functional properties to HFSCs, they are commonly referred to as HFSCs and will be referred to here as such. The term “induced hair follicle stem cell” (iHFSC) encompasses HFSCs or HFPCs that arise from non-HFS cells by experimental manipulation. iHFSCs exhibit the same phenotypic and functional properties as HFSCs or HFPCs, as defined above. Unless otherwise indicated, as used herein the term iHFSC refers to a non-auditory iHFSC.

The term “hair unit” encompasses a combination of epidermal cells and dermal cells. Epidermal cells may contain HFSCs, HFPCs, and/or iHFCs. Dermal cells may comprise dermal papilla cells, dermal sheath cells, other cell types known to compose the dermis or cells that are functionally equivalent to these cell types. The hair unit may produce hair in a culture dish. The hair unit may also produce hair upon transplantation in vivo.

HFSCs express one or more marker(s) including E-Cadherin (ECAD) (also referred to as Cadherin 1 (CDH1)), Keratin 15 (KRT15), Kruppel Like Factor 5 (KLF5), HR Lysine Demethylase and Nuclear Receptor Corepressor (HR), SRY-Box Transcription Factor 9 (SOX9), Transcription Factor AP-2 Alpha (TFAP2A), Integrin Subunit Alpha 6 (ITGA6), BAF Chromatin Remodeling Complex Subunit BCL11B (CTIP2), Epithelial Splicing Regulatory Protein 1 (ESRP1), T-box Transcription Factor 1 (TBX1), and other markers known to those skilled in the art. In some embodiments, HFSCs express one, two, three, four, five, six, seven, eight, nine, or all of the markers selected from the group consisting of: ECAD, KRT15, KLF5, HR, SOX9, TFAP2A, ITGA6, CTIP2, ESRP1 and TBX1. In some embodiments, expression levels of one or more of the markers selected from the group consisting of: ECAD, KRT15, KLF5, HR, SOX9, TFAP2A, ITGA6, CTIP2, ESRP1 and TBX1 are modulated over time and/or depending on culture conditions. For example, HFSCs can express at least two-fold more, at least 3-fold more, at least 4-fold more, at least 5-fold more, at least 6-fold more, at least 7-fold more, at least 8-fold more, at least 9-fold more, or at least 10 fold more of one or more of the markers selected from the group consisting of ECAD, KRT15, KLF5, HR, SOX9, TFAP2A, ITGA6, CTIP2, ESRP1, and TBX1, as compared to a population of non-HFS cells. In an embodiment, expression of the one or more markers is measured using immunofluorescent staining (e.g., flow cytometry or immunohistochemistry), or western blot analysis. In a preferred embodiment, expression of the one or more markers is measured using flow cytometry. In some embodiments, expression levels of mRNA encoding the one or more markers is measured. For example, in some embodiments, mRNA levels are measured using, quantitative polymerase chain reaction (qPCR), reverse transcription polymerase chain reaction (RT PCR), RNA-Seq, or northern blot analysis. In a preferred embodiment, mRNA levels of the one or more markers is measured using qPCR. In addition, HFSCs are mitotic, and so can incorporate BrdU into their DNA or exhibit evidence of cell division as known to those skilled in the art, such as cell division that occurs during cell passaging. In some embodiments, HFSCs maintain expression of one or more of the markers selected from the group consisting of ECAD, KRT15, KLF5, HR, SOX9, TFAP2A, ITGA6, CTIP2, ESRP1, and TBX1, over at least one cell passage, for example, at least one cell passage, at least two cell passages, at least 3 cell passages, at least 4 cell passages, or at least 5 cell passages.

### Non-Hair Follicle Stem Cells

As used herein, the term “non-hair follicle stem (HFS) cell” encompasses any cell in an organism that cannot give rise to cells composing hair follicles under normal physiological conditions. This term may encompass other stem cells, hematopoietic cells, epithelial cells, dermal cells, or any other cell type that cannot give rise to hair follicle cells under normal physiological conditions absent genetic manipulation or wounding (for example, cutting, burning or scarring). The non-HFS cells may be from any mammal, including humans, primates, domestic and farm animals, and zoo, laboratory or pet animals, such as dogs, cats, cattle, horses, sheep, pigs, goats, rabbits, rats, mice etc. In some embodiments, non-HFS cells are human or murine cells and are dermal cells (e.g., fibroblasts, smooth muscle cells, or connective tissue cells), epithelial cells, adipocytes, or hematopoietic cells.

In some embodiments, non-HFS cells may be established cell lines or they may be primary cells, where “primary cells,” “primary cell lines,” and “primary cultures” are used interchangeably herein to refer to cells and cells cultures that have been derived from a subject and allowed to grow in vitro for a limited number of passages. For example, primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro. The non-HFS cells may be isolated from fresh or frozen cells, which may be from a neonate, a juvenile or an adult, and from tissues including skin, muscle, bone marrow, peripheral blood, umbilical cord blood, spleen, bladder, liver, pancreas, lung, intestine, stomach, adipose, and other differentiated tissues. The tissue may be obtained by biopsy or apheresis from a live donor or obtained from a dead or dying donor within about 48 hours of death, or freshly frozen tissue, tissue frozen within about 12 hours of death and maintained at below about −20° C., usually at about liquid nitrogen temperature (−190° C.) indefinitely. For isolation of cells from tissue, an appropriate solution may be used for dispersion or suspension. Such a solution will generally be a balanced salt solution, e.g., normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

### Transdifferentiation and Hair Follicle Stem Cell Reprogramming System

The term “transdifferentiation” refers to the deliberate induction of a transition of a cell or group of cells to a different cell type using genetic or biochemical manipulations such as introduction of an exogenously derived gene or protein into the cell. This is distinct from the normal processes of differentiation or de-differentiation, which may be artificially induced through standard cell culture procedures or treatment with growth factors or other signaling molecules.

The terms “hair follicle stem cell reprogramming factors” or “HFSCR factors” refer to one or more, i.e. a cocktail, of biologically active factors that act on a non-HFS cell to promote reprogramming, i.e. transdifferentiation, of the targeted cell into a non-auditory iHFSC. As used herein, the term “HFSCR system” refers to reagents and culture conditions that promote the reprogramming, i.e. transdifferentiation, of non-HFS cells to non-auditory iHFSCs where the non-HFS cells may be somatic cells or may be pluripotent cells. A HFSCR system comprises one or more, i.e. a cocktail, of non HFS cell-to-HFSCR factors. A HFSCR system may also optionally comprise other reagents, such as agents that promote cell reprogramming, agents that promote the survival and differentiation of HFSCs, agents that promote the differentiation of subtypes of HFSCs, agents that promote the survival and differentiation of HFPCs, agents that promote the differentiation of subtypes of HFPCs, and the like, as known in the art. A HFSCR system does not induce a non-HFS cell to become pluripotent, e.g., an induced pluripotent stem cell (iPSC), in the course of conversion into non-auditory iHFSCs. In other words, a HFSCR system induces the transdifferentiation of non-HFS cells of one lineage into non-auditory iHFSCs, or induces pluripotent cells to become non-auditory iHFSCs.

In some embodiments, HFSCR systems comprise conditions that induce the conversion of non-HFS cells into non-auditory iHFSCs from a subject; or induce the conversion of a pluripotent cell into an iHFSC. In some embodiments, the non-HFS cells are contacted in vitro with the HFSCR system comprising of one or more non-HFS cell-to-HFSC reprogramming factors (HFSCR factors). In some embodiments, the one or more HFSCR factors are provided as nuclear acting polypeptides. In other words, the subject cells are contacted with HFSCR polypeptides that act in the nucleus.

To promote transport of HFSCR polypeptides across the cell membrane, HFSCR polypeptide sequences may be fused to a polypeptide permeant domain. A number of permeant domains are known in the art and may be used in the nuclear acting polypeptides of the present invention, including peptides, peptidomimetics, and non-peptide carriers. For example, a permeant peptide may be derived from the third alpha helix of Drosophila melanogaster transcription factor Antennapedia, referred to as penetratin, which comprises the amino acid sequence RQIKIWFQNRRMKWKK (SEQ ID NO: 1). As another example, the permeant peptide comprises the HIV-1 tat basic region amino acid sequence, which may include, for example, amino acids 49-57 of the naturally-occurring tat protein (SEQ ID NO: 2). Other permeant domains include poly-arginine motifs, for example, the region of amino acids 34-50 of HIV-1 rev protein (SEQ ID NO:3), nona-arginine (SEQ ID NO:4), octa-arginine (SEQ ID NO:5), and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24): 13003-8: published U.S. Patent applications 20030220334; 20030083256; 20030032593; and 20030022831, herein specifically incorporated by reference for the teachings of translocation peptides and peptoids). The nona-arginine (R9; SEQ ID NO:4) sequence is one of the more efficient PTDs that have been characterized (Wender et al. 2000; Uemura et al. 2002).

The HFSCR polypeptides may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. Other methods of preparing polypeptides in a cell-free system include, for example, those methods taught in U.S. Application Ser. No. 61/271,000.

The HFSCR polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein. HFSCR polypeptides may be produced recombinantly not only directly, but also as a fusion polypeptide with a heterologous polypeptide, e.g., a polypeptide having a specific cleavage site at the N-terminus of the mature protein or polypeptide. Expression vectors usually contain a selection gene, also termed a selectable marker. This gene encodes a protein necessary for the survival or growth of transformed host cells grown in a selective culture medium.

Following purification by commonly known methods in the art, HFSCR polypeptides are provided to the subject cells by standard protein transduction methods. In some cases, the protein transduction method includes contacting cells with a composition containing a carrier agent and at least one purified HFSCR polypeptide. Examples of suitable carrier agents and methods for their use include, but are not limited to, commercially available reagents such as Chariot™ (Active Motif, Inc., Carlsbad, Calif.) described in U.S. Pat. No. 6,841,535: Bioport™ (Gene Therapy Systems, Inc., San Diego, Calif.), GenomeONE (Cosmo Bio Co., Ltd., Tokyo, Japan), and ProteoJuice™ (Novagen, Madison, Wis.), or nanoparticle protein transduction reagents.

In other embodiments, the one or more HFSCR factors are nucleic acids (e.g., polynucleotides) encoding HFSCR polypeptides, i.e. HFSCR nucleic acids. Nucleic acids can be deoxyribonucleic acids (DNA), ribonucleic acids (RNA) or functionally similar derivatives. Vectors used for providing HFSCR nucleic acids to the subject cells will typically comprise suitable promoters for driving the expression, that is, transcriptional activation, of the nucleic acids. This may include ubiquitously acting promoters, for example, the CMV-β-actin promoter, or inducible promoters, such as promoters that are active in particular cell populations or that respond to the presence of drugs such as tetracycline. By transcriptional activation, it is intended that transcription will be increased above basal levels in the target cell by at least about 10-fold, by at least about 100-fold, more usually by at least about 1000-fold. In addition, vectors used for providing the nucleic acids may include genes that must later be removed, e.g., using a recombinase system such as Cre/Lox, or genes that cause the cells that express them to be destroyed, e.g., by including genes that allow selective toxicity such as herpesvirus TK, bcl-xs, etc.

HFSCR nucleic acids may be provided directly to the subject cells. In other words, the cells are contacted with vectors comprising HFSCR nucleic acids such that the vectors are taken up by the cells. Methods for contacting cells with nucleic acid vectors, such as electroporation, calcium chloride transfection, and lipofection, are well known in the art. Vectors that deliver nucleic acids in this manner are usually maintained episomally, e.g., as plasmids or minicircle DNAs.

Alternatively, the nucleic acid may be provided to the subject cells via a virus. In other words, the cells are contacted with viral particles comprising the HFSCR nucleic acids. Retroviruses, for example, lentiviruses, are particularly suitable for such methods. Commonly used retroviral vectors are “defective,” i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g., MMLV, are capable of infecting most murine and rat cell types, and are generated by using ecotropic packaging cell lines such as BOSC23 (Pear et al. (1993) Proc. Natl. Acad. Sci. U.S.A. 90:8392-8396). Retroviruses bearing amphotropic envelope protein, e.g., 4070A (Danos et al., supra.), are capable of infecting most mammalian cell types, including human, dog and mouse, and are generated by using amphotropic packaging cell lines such as PA12 (Miller et al. (1985) Mol. Cell. Biol. 5:431-437): PA317 (Miller et al. (1986) Mol. Cell. Biol. 6:2895-2902): GRIP (Danos et al. (1988) Proc. Natl. Acad. Sci. U.S.A. 85:6460-6464). Retroviruses packaged with xenotropic envelope protein, e.g., AKR env, are capable of infecting most mammalian cell types, except murine cells. The appropriate packaging cell line may be used to ensure that the subject cells are targeted by the packaged viral particles. Methods of introducing the retroviral vectors comprising HFSCR nucleic acids into packaging cell lines and of collecting the viral particles that are generated by the packaging lines are well known in the art.

The effective amount of a HFSCR system that may be used to contact the non-HFS cells is an amount that induces at least 0.01% of the cells of the culture to increase expression of one or more genes known in the art to become more highly expressed upon the acquisition of a HFSC fate, e.g., KRT15, ECAD (CDH1), KLF5, HR, SOX9, TFAP2A, ITGA6, CTIP2, ESRP1, and TBX1.

An effective amount is the amount that induces an increase in expression of these genes that is, e.g., about 1.5 fold, 2 fold, 3 fold, 4 fold, 6 fold, or 10 fold greater than the level of expression observed in the absence of the HFSCR system. The level of gene expression can be readily determined by any of a number of well-known methods in the art, e.g., by measuring RNA levels by methods such as, but not limited to, RT-PCR, quantitative RT-PCR, RNA-Seq, and Northern blot; and by measuring protein levels by methods such as, but not limited to, Western blot, ELISA, and fluorescence activated cell sorting.

It is noted here that the contacted non-HFS cells do not need to be cultured under methods known in the art to promote pluripotency in order to be converted into iHFSCs. By pluripotency, it is meant that the cells have the ability to differentiate into all types of cells in an organism.

For the HFSCR system, the efficiency of reprogramming may be determined by assaying the number of iHFSCs that develop in the cell culture, e.g. by assaying the number of cells that express genes that are expressed by HFSCs, e.g., KLF5, HR, SOX9, TFAP2A, ITGA6, CTIP2, ESRP1, and TBX1.

For the HFSCR system, following the methods of the invention, the contacted non-HFS cells will be converted into iHFSCs or HFPCs at an efficiency of reprogramming/efficiency of conversion that is at least about 0.01% of the total number of non-HFS cells cultured initially, e.g., about 0.01%, 0.05%, 0.1%, 0.5%, 1%, 2%, 3%, 4%, 5%, 6%, 7%, 8%, 9%, 10%, 12%, 14%, 16%, 20% or more.

At times, depending on the age of the donor, the origin of the tissue, or the culture conditions, higher efficiencies may be achieved. This efficiency of reprogramming is an enhanced efficiency over that which may be observed in the absence of HFSCR system(s). By enhanced, it is meant that the non-HFS cells cultures have the ability to give rise to the desired cell type that is at least 150% greater than the ability of a non-HFS cell culture that was not contacted with the HFSCR factor(s), e.g., at least 150%, at least 200%, at least 300%, at least 400%, at least 600%, at least 800%, at least 1000%, or at least 2000% of the ability of the uncontacted population. In other words, for cells treated with the HFSCR system, the culture of non-HFS cells produces at least 1.5 fold, at least 2-fold, at least 3-fold, at least 4-fold, at least 6-fold, at least 10-fold, at least 20-fold, at least 30-fold, at least 50-fold, at least 100-fold, or at least 200-fold the number of iHFSCs that are produced by a population of non-HFS cells that are not contacted with the HFSCR system.

In some cases, genes may be introduced into the non-HFS cells or the cells derived therefrom, i.e. iHFSCs or differentiated progeny cells, prior to transferring to a subject for a variety of purposes, for example, but not limited to, replacing genes having a loss of function mutation (e.g., loss of function mutations in ECAD, KRT15, KLF5, HR, SOX9, TFAP2A, ITGA6, CTIP2, ESRP1, and/or TBX1), or providing marker genes (e.g., Green Fluorescent Protein, or antibiotic resistance genes). Alternatively, vectors are introduced that express antisense mRNA or ribozymes, thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as Bcl-2. Various techniques known in the art may be used to introduce nucleic acids into the target cells, e.g., electroporation, calcium precipitated DNA, fusion, transfection, lipofection, infection and the like, as discussed above. The particular manner in which the nucleic acids are introduced is not critical to the practice of the invention.

To confirm that non-HFS cells or the cells derived therefrom, have been genetically modified, i.e. transdifferentiated to iHFSCs or differentiated progeny cells, various techniques may be employed. The genome, transcriptome, or proteome of the cells may be restricted and used with or without amplification. Polymerase chain reaction; gel electrophoresis; restriction analysis; Southern, Northern, and Western blots; sequencing; or the like, may all be employed to confirm genetic manipulation. The cells may be grown under various conditions to ensure that the cells are capable of maturation to all of the hair follicle lineages. Various tests in vitro and in vivo may be employed to ensure that the iHFSC or differentiated progeny cell phenotype of the derived cells has been maintained.

### Hair Follicle Stem Cell Reprogramming (HFSCR) Factors

The following describes the HFSCR factors for the HFSCR system described in the previous section.

HFSCR factors are biologically active factors that act on a cell to alter transcription so as to convert the cell into a HFSC, i.e. an iHFSC. HFSCR factors are provided to somatic or pluripotent cells in the context of an HFSCR system. Examples of HFSCR factors include a Lhx agent, an NF-I agent, a TFAP2 agent, an Irx agent, a p53 agent, and a Myc agent.

As used herein, the term “functional fragment” refers to a portion of a full-length protein that maintains the ability to perform a cellular function of the full-length protein and/or interact with a protein binding partner of the full-length protein.

The term Lhx agent is used to refer to Lhx (also called LIM homeobox) polypeptides, functional fragments thereof, and the nucleic acids that encode them. The term Lhx agent may also refer to polypeptides of Lhx-related proteins or proteins that regulate Lhx activity and the nucleic acids (e.g., polynucleotides) that encode them. In some embodiments, an Lhx polypeptide or functional fragment thereof is fused to a permeant domain. Lhx agents may also refer to small molecules that modulate Lhx expression and/or activity. Lhx polypeptides are homeodomain containing transcription factors with a LIM domain, a cysteine-rich zinc binding domain. The terms “Lhx gene product,” “Lhx polypeptide,” and “Lhx protein” are used interchangeably herein to refer to native sequence Lhx polypeptides, Lhx polypeptide variants, Lhx polypeptide fragments and chimeric Lhx polypeptides that can modulate transcription. Native sequence Lhx polypeptides include the proteins Lhx1 (also called LIM1: GenBank Accession Nos. NM 005568.2 and NP 005559.2): Lhx2 (GenBank Accession Nos. NM 004789.3 and NP 004780.3): Lhx3 (GenBank Accession Nos. NM_178138.3 and NP 835258.1 (isoform a), and NM_014564.2 and NP 055379.1 (isoform b)): Lhx4 (GenBank Accession Nos. NM_033343.2 and NP 203129.1); Lhx5 (GenBank Accession Nos. NM_022363.2 and NP 071758.1); Lhx6 (GenBank Accession Nos. NM_014368.3 and NP 055183.2 (isoform 1), and NM_199160.2 and NP_954629.2 (isoform 2)); Lhx7 (also called Lhx8: GenBank Accession Nos. NM_001001933.1 and NP_001001933.1); and Lhx9 (GenBank Accession Nos. NM_020204.2 and NP_064589.2 (isoform 1), and NM_001014434.1 and NP_001014434.1 (isoform 2)). Lhx polypeptides, e.g., those that are at least 50%, 55%, 60%, 65%, 70%, 75%, 80%, 85%, 90%, 91%, 92%, 95%, 97%, 99%, or are 100% identical to the sequence provided in the GenBank Accession Nos. above find use as reprogramming factors in the present invention, as do nucleic acids encoding these polypeptides or their transcriptionally active domains and vectors comprising these nucleic acids. In some embodiments, the Lhx agent is a Lhx2 agent. In certain embodiments, the Lhx agent is a polynucleotide encoding a Lhx2 polypeptide.

The term NF-I agent is used to refer to NF-I (Nuclear factor I) polypeptides, functional fragments thereof, and the nucleic acids that encode them. The term NF-I agent may also refer to polypeptides of NF-I-related proteins or proteins that regulate NF-I activity and the nucleic acids (e.g., polynucleotides) that encode them. In some embodiments, an NF-I polypeptide or functional fragment thereof is fused to a permeant domain. NF-I agents may also refer to small molecules that modulate NF-I expression and/or activity. NF-I polypeptides are members of the NF-I (Nuclear factor I) family of DNA binding proteins. The terms “NF-I gene product,” “NF-I polypeptide,” and “NF-I protein” are used interchangeably herein to refer to native sequence NF-I polypeptides, NF-I polypeptide variants, NF-I polypeptide fragments and chimeric NF-I polypeptides that can modulate transcription. Native sequence NF-I polypeptides include the proteins NFIA (GenBank Accession Nos. NM_001134673.4 and NP_001128145.1 (isoform 1), GenBank Accession Nos. NM_005595.5 and NP_005586.1 (isoform 2), GenBank Accession Nos. NM_001145511.2 and NP_001138983.1 (isoform 3), and GenBank Accession Nos. NM_001145512.2 and NP_001138984.1 (isoform 4)): NFIB (GenBank Accession Nos. NM_001190737.2 and NP_001177666.1 (isoform 1), GenBank Accession Nos. NM_001190738.2 and NP_001177667.1 (isoform 2), GenBank Accession Nos. NM_005596.3 and NP_005587.2 (isoform 3), GenBank Accession Nos. NM_001282787.2 and NP_001269716.1 (isoform 4), GenBank Accession Nos. NM_001369458.1 and NP_001356387.1 (isoform 5), GenBank Accession Nos. NM_001369459.1 and NP_001356388.1 (isoform 6), GenBank Accession Nos. NM_001369460.1 and NP_001356389.1 (isoform 7), GenBank Accession Nos. NM_001369461.1 and NP_001356390.1 (isoform 8), GenBank Accession Nos. NM_001369462.1 and NP_001356391.1 (isoform 9), GenBank Accession Nos. NM_001369463.1 and NP_001356392.1 (isoform 10), GenBank Accession Nos. NM_001369464.1 and NP_001356393.1 (isoform 11), GenBank Accession Nos. NM_001369465.1 and NP_001356394.1 (isoform 12), GenBank Accession Nos. NM_001369466.1 and NP_001356395.1 (isoform 13), GenBank Accession Nos. NM_001369467.1 and NP_001356396.1 (isoform 14), GenBank Accession Nos. NM_001369468.1 and NP_001356397.1 (isoform 15), GenBank Accession Nos. NM_001369469.1 and NP_001356398.1 (isoform 16), GenBank Accession Nos. NM_001369470.1 and NP_001356399.1 (isoform 17), GenBank Accession Nos. NM_001369471.1 and NP_001356400.1 (isoform 18), GenBank Accession Nos. NM_001369472.1 and NP_001356401.1 (isoform 19), GenBank Accession Nos. NM_001369473.1 and NP_001356402.1 (isoform 20), GenBank Accession Nos. NM_001369474.1 and NP_001356403.1 (isoform 21), GenBank Accession Nos. NM_001369475.1 and NP_001356404.1 (isoform 22), GenBank Accession Nos. NM_001369476.1 and NP_001356405.1 (isoform 23), GenBank Accession Nos. NM_001369477.1 and NP_001356406.1 (isoform 24), GenBank Accession Nos. NM_001369478.1 and NP_001356407.1 (isoform 25), GenBank Accession Nos. NM_001369479.1 and NP_001356408.1 (isoform 26), GenBank Accession Nos. NM_001369480.1 and NP_001356409.1 (isoform 27), GenBank Accession Nos. NM_001369481.1 and NP_001356410.1 (isoform 28)): NFIC (GenBank Accession Nos. NM_001245002.2 and NP_001231931.1 (isoform 1), GenBank Accession Nos. NM_205843.3 and NP_995315.1 (isoform 2), GenBank Accession Nos. NM_001245004.2 and NP_001231933.1 (isoform 3), GenBank Accession Nos. NM_001245005.2 and NP_001231934.1 (isoform 4), and GenBank Accession Nos. NM_005597.4 and NP_005588.2 (isoform 5)); and NFIX (GenBank Accession Nos. NM_001271043.2 and NP_001257972.1 (isoform 1), GenBank Accession Nos. NM_002501.4 and NP_002492.2 (isoform 2), GenBank Accession Nos. NM_001271044.3 and NP_001257973.1 (isoform 3), GenBank Accession Nos. NM_001365902.3 and NP_001352831.1 (isoform 4), GenBank Accession Nos. NM_001365982.2 and NP_001352911.1 (isoform 5), GenBank Accession Nos. NM_001365983.2 and NP_001352912.1 (isoform 6), GenBank Accession Nos. NM_001365984.2 and NP_001352913.1 (isoform 7), GenBank Accession Nos. NM_001365985.2 and NP_001352914.1 (isoform 8), GenBank Accession Nos. NM_001378404.1 and NP_001365333.1 (isoform 9), and GenBank Accession Nos. NM_001378405.1 and NP_001365334.1 (isoform 10)). NF-I polypeptides, e.g., those that are at least 50%, 55%, 60%, 65%, 70%, 75%, 80%, 85%, 90%, 91%, 92%, 95%, 97%, 99%, or are 100% identical to the sequence provided in the GenBank Accession Nos. above find use as reprogramming factors in the present invention, as do nucleic acids encoding these polypeptides or their transcriptionally active domains and vectors comprising these nucleic acids. In certain embodiments, the NF-I agent is a NFIB agent. In some embodiments, the NF-I agent is a polynucleotide encoding a NFIB polypeptide.

The term TFAP2 agent is used to refer to TFAP2 (Transcription Factor AP-2 Alpha 2) polypeptides, functional fragments thereof, and the nucleic acids that encode them. The term TFAP2 agent may also refer to polypeptides of TFAP2-related proteins or proteins that regulate TFAP2 activity and the nucleic acids (e.g., polynucleotides) that encode them. In some embodiments, a TFAP2 polypeptide or functional fragment thereof is fused to a permeant domain. TFAP2 agents may also refer to small molecules that modulate TFAP2 expression and/or activity. TFAP2 polypeptides are members of the AP-2 (Activating Protein 2) family of transcription factors. The terms “TFAP2 gene product,” “TFAP2 polypeptide,” and “TFAP2 protein” are used interchangeably herein to refer to native sequence TFAP2 polypeptides, TFAP2 polypeptide variants, TFAP2 polypeptide fragments and chimeric TFAP2 polypeptides that can modulate transcription. Native sequence TFAP2 polypeptides include the proteins TFAP2A (GenBank Accession Nos. NM_001372066.1 and NP_001358995.1 (isoform a), GenBank Accession Nos. NM_001032280.3 and NP_001027451.1 (isoform b), GenBank Accession Nos. NM_001042425.3 and NP_001035890.1 (isoform c)); TFAP2B (GenBank Accession Nos. NM_003221.3 and NP_003212.2): TFAP2C (GenBank Accession Nos. NM_003222.3 and NP_003213.1); TFAP2D (GenBank Accession Nos. NM_172238.3 and NP_758438.2); and TFAP2E (GenBank Accession Nos. NM_178548.3 and NP_848643.2). TFAP2 polypeptides, e.g., those that are at least 50%, 55%, 60%, 65%, 70%, 75%, 80%, 85%, 90%, 91%, 92%, 95%, 97%, 99%, or are 100% identical to the sequence provided in the GenBank Accession Nos. above find use as reprogramming factors in the present invention, as do nucleic acids encoding these polypeptides or their transcriptionally active domains and vectors comprising these nucleic acids. In certain embodiments, the TFAP2 agent is a TFAP2A agent. In some embodiments, the TFAP2 agent is a polynucleotide encoding a TFAP2A polypeptide.

The term Irx agent is used to refer to Irx (Iroquois homeobox factor) polypeptides, functional fragments thereof, and the nucleic acids that encode them. The term Irx agent may also refer to polypeptides of Irx-related proteins or proteins that regulate Irx activity and the nucleic acids (e.g., polynucleotides) that encode them. In some embodiments, an Irx polypeptide or functional fragment thereof is fused to a permeant domain. Irx agents may also refer to small molecules that modulate Irx expression and/or activity. Irx polypeptides are members of the Irx (Iroquois homeobox factor) transcription factor family. The terms “Irx gene product,” “Irx polypeptide,” and “Irx protein” are used interchangeably herein to refer to native sequence Irx polypeptides, Irx polypeptide variants, Irx polypeptide fragments and chimeric Irx polypeptides that can modulate transcription. Native sequence Irx polypeptides include the proteins Irx1 (GenBank Accession Nos. NM_024337.3 and NP_077313.3): Irx2 (GenBank Accession Nos. NM_033267.4 and NP_150366.1 (variant 1) and GenBank Accession Nos NM_001134222.2 and NP_001127694.1 (variant 2)): Irx3 (GenBank Accession Nos. NM_024336.2 and NP_077312.2): Irx4 (GenBank Accession Nos. NM_001278632.1 and NP_001265561.1 (variant 1), GenBank Accession Nos. NM_001278633.1 and NP_001265562.1 (variant 2), GenBank Accession Nos. NM_001278634.2 and NP_001265563.1 (variant 3), GenBank Accession Nos. NM_001278635.2 and NP_001265564.1 (variant 4), and GenBank Accession Nos. NM_016358.3 and NP_057442.1 (variant 5)); Irx5 (GenBank Accession Nos. NM_005853.5 and NP_005844.4 (variant 1) and GenBank Accession Nos. NM_001252197.1 and NP_001239126.1 (variant 2)); Irx6 (GenBank Accession Nos. NM_024335.2 and NP_077311.2). Irx polypeptides, e.g., those that are at least 50%, 55%, 60%, 65%, 70%, 75%, 80%, 85%, 90%, 91%, 92%, 95%, 97%, 99%, or are 100% identical to the sequence provided in the GenBank Accession Nos. above find use as reprogramming factors in the present invention, as do nucleic acids encoding these polypeptides or their domains that affect transcription and vectors comprising these nucleic acids. In certain embodiments, the Irx agent is a Irx4 agent. In some embodiments, the Irx agent is a polynucleotide encoding an Irx4 polypeptide.

The term p53 agent is used to refer to p53 polypeptides, functional fragments thereof, and the nucleic acids that encode them. The term p53 agent may also refer to polypeptides of p53-related proteins or proteins that regulate p53 activity and the nucleic acids (e.g., polynucleotides) that encode them. In some embodiments, a p53 polypeptide or functional fragment thereof is fused to a permeant domain. p53 agents may also refer to small molecules that modulate p53 expression and/or activity. p53 polypeptides are members of the p53 transcription factor family. The terms “p53 gene product,” “p53 polypeptide,” and “p53 protein” are used interchangeably herein to refer to native sequence p53 polypeptides, p53 polypeptide variants, p53 polypeptide fragments and chimeric p53 polypeptides that can modulate transcription. Native sequence p53 polypeptides include the proteins TP53 (GenBank Accession Nos. NM_000546.6 and NP_000537.3 (isoform a), GenBank Accession Nos. NM_001126114.3 and NP_001119586.1 (isoform b), GenBank Accession Nos. NM_001126113.3 and NP_001119585.1 (isoform c), GenBank Accession Nos. NM_001126115.2 and NP_001119587.1 (isoform d), GenBank Accession Nos. NM_001126116.2 and NP_001119588.1 (isoform e), GenBank Accession Nos. NM_001126117.2 and NP_001119589.1 (isoform f), GenBank Accession Nos. NM_001126118.2 and NP_001119590.1 (isoform g), GenBank Accession Nos. NM_001276695.3 and NP_001263624.1 (isoform h), GenBank Accession Nos. NM_001276696.3 and NP_001263625.1 (isoform i), GenBank Accession Nos. NM_001276697.3 and NP_001263626.1 (isoform j), GenBank Accession Nos. NM_001276698.3 and NP_001263627.1 (isoform k), and GenBank Accession Nos. NM_001276699.3 and NP_001263628.1 (isoform 1)): TP63 (GenBank Accession Nos. NM_003722.5 and NP_003713.3 (isoform 1), GenBank Accession Nos. NM_001114978.2 and NP_001108450.1 (isoform 2), GenBank Accession Nos. NM_001114979.2 and NP_001108451.1 (isoform 3), GenBank Accession Nos. NM_001114980.2 and NP_001108452.1 (isoform 4), GenBank Accession Nos. NM_001114981.2 and NP_001108453.1 (isoform 5), GenBank Accession Nos. NM_001114982.2 and NP_001108454.1 (isoform 6), GenBank Accession Nos. NM_001329144.2 and NP_001316073.1 (isoform 7), GenBank Accession Nos. NM_001329145.2 and NP_001316074.1 (isoform 8), GenBank Accession Nos. NM_001329146.2 and NP_001316075.1 (isoform 9), GenBank Accession Nos. NM_001329148.2 and NP_001316077.1 (isoform 10), GenBank Accession Nos. NM_001329149.2 and NP_001316078.1 (isoform 11), GenBank Accession Nos. NM_001329150.2 and NP_001316079.1 (isoform 12), and GenBank Accession Nos. NM_001329964.2 and NP_001316893.1 (isoform 13)); and TP73 (GenBank Accession Nos. NM_005427.4 and NP_005418.1 (isoform a), GenBank Accession Nos. NM_001126240.3 and NP_001119712.1 (isoform b), GenBank Accession Nos. NM_001126241.3 and NP_001119713.1 (isoform c), GenBank Accession Nos. NM_001126242.3 and NP_001119714.1 (isoform d), GenBank Accession Nos. NM_001204189.2 and NP_001191118.1 (isoform e), GenBank Accession Nos. NM_001204190.2 and NP_001191119.1 (isoform f), GenBank Accession Nos. NM_001204191.2 and NP_001191120.1 (isoform g), GenBank Accession Nos. NM_001204184.2 and NP_001191113.1 (isoform h), GenBank Accession Nos. NM_001204185.2 and NP_001191114.1 (isoform i), GenBank Accession Nos. NM_001204186.2 and NP_001191115.1 (isoform j), GenBank Accession Nos. NM_001204187.2 and NP_001191116.1 (isoform k), GenBank Accession Nos. NM_001204188.2 and NP_001191117.1 (isoform 1), GenBank Accession Nos. NM_001204192.2 and NP_001191121.1 (isoform m)). p53 polypeptides, e.g., those that are at least 50%, 55%, 60%, 65%, 70%, 75%, 80%, 85%, 90%, 91%, 92%, 95%, 97%, 99%, or are 100% identical to the sequence provided in the GenBank Accession Nos. above find use as reprogramming factors in the present invention, as do nucleic acids encoding these polypeptides or their domains that affect transcription and vectors comprising these nucleic acids. In some embodiments, the p53 agent is a TP63 agent. In some embodiments, the p53 agent is a polynucleotide encoding a TP63 polypeptide.

The term Myc agent is used to refer to Myc polypeptides, functional fragments thereof, and the nucleic acids that encode them. The term Myc agent may also refer to polypeptides of Myc-related proteins or proteins that regulate Myc activity and the nucleic acids (e.g., polynucleotides) that encode them. In some embodiments, a Myc polypeptide or functional fragment thereof is fused to a permeant domain. Myc agents may also refer to small molecules that modulate Myc expression and/or activity. Myc polypeptides are members of the Myc transcription factor family. The terms “Myc gene product,” “Myc polypeptide,” and “Myc protein” are used interchangeably herein to refer to native sequence Myc polypeptides, Myc polypeptide variants, Myc polypeptide fragments and chimeric Myc polypeptides that can modulate transcription. Native sequence Myc polypeptides include the proteins MYC (GenBank Accession Nos. NM_002467.6 and NP_002458.2 (isoform 1), and GenBank Accession Nos. NM_001354870.1 and NP_001341799.1 (isoform 2)); MYCL (GenBank Accession Nos. NM_001033081.3 and NP_001028253.1 (isoform 1), GenBank Accession Nos. NM_005376.5 and NP_005367.2 (isoform 2), and GenBank Accession Nos. NM_001033082.3 and NP_001028254.2 (isoform 3)); and MYCN (GenBank Accession Nos. NM_001293228.2 and NP_001280157.1), GenBank Accession Nos. NM_001293231.2 and NP_001280160.1 (isoform 2), and GenBank Accession Nos. NM_001293233.2 and NP_001280162.1 (isoform 3)). Myc polypeptides, e.g., those that are at least 50%, 55%, 60%, 65%, 70%, 75%, 80%, 85%, 90%, 91%, 92%, 95%, 97%, 99%, or are 100% identical to the sequence provided in the GenBank Accession Nos. above find use as reprogramming factors in the present invention, as do nucleic acids encoding these polypeptides or their domains that affect transcription and vectors comprising these nucleic acids. In certain embodiments, the Myc agent is a MYC agent. In some embodiments, the Myc agent is a polynucleotide encoding a MYC polypeptide.

In some embodiments, only one HFSCR factor is provided, e.g., a p53 agent, a NF-I agent, a Lhx agent, a TFAP2 agent, an Irx agent, or a Myc agent. For example, in certain embodiments, only one HFSCR factor is provided selected from a group consisting of a TP63 agent, an NFIB agent, an LHX2 agent, a TFAP2A agent, an IRX4 agent, and a MYC agent. In some embodiments, only one HFSCR factor is provided selected from a group consisting of a polynucleotide encoding a TP63 polypeptide. a polynucleotide encoding a NFIB polypeptide, a polynucleotide encoding a LHX2 polypeptide, a polynucleotide encoding a TFAP2A polypeptide, a polynucleotide encoding an IRX4 polypeptide, and a polynucleotide encoding a MYC polypeptide.

In some embodiments, a set of at least two agents is provided, e.g., a p53 agent and an NF-I agent, a p53 agent and a Lhx agent, a p53 agent and a TFAP2 agent, a p53 agent and an Irx agent, a p53 agent and a Myc agent, a NF-I agent and a Lhx agent, a NF-I agent and a TFAP2 agent, a NF-I agent and a Lhx agent, a NF-I agent and an Irx agent, a NF-I agent and a Myc agent, an LHX2 agent and a TFAP2 agent, an LHX2 agent and a Irx agent, an LHX2 agent and a Myc agent, a TFAP2 agent and an Irx agent, a TFAP2 agent and a Myc agent, or an Irx agent and a Myc agent. In some embodiments, a set of at least two agents is provided selected from the group consisting of a TP63 agent and an NFIB agent, a TP63 agent and a LHX2 agent, a TP63 agent and a TFAP2A agent, a TP63 agent and an IRX4 agent, a TP63 agent and a MYC agent, a NFIB agent and a LHX2 agent, a NFIB agent and a TFAP2A agent, a NFIB agent and a LHX2 agent, a NFIB agent and an IRX4 agent, a NFIB agent and a MYC agent, an LHX2 agent and a TFAP2A agent, an LHX2 agent and a IRX4 agent, an LHX2 agent and a MYC agent, a TFAP2A agent and an IRX4 agent, a TFAP2A agent and a MYC agent, and an IRX4 agent and a MYC agent. In some embodiments, a set of at least two agents is provided selected from the group consisting of a polynucleotide encoding a TP63 polypeptide and a polynucleotide encoding a NFIB polypeptide, a polynucleotide encoding a TP63 polypeptide and a polynucleotide encoding a LHX2 polypeptide, a polynucleotide encoding a TP63 polypeptide and a polynucleotide encoding a TFAP2A polypeptide, a polynucleotide encoding a TP63 polypeptide and a polynucleotide encoding an IRX4 polypeptide, a polynucleotide encoding a TP63 polypeptide and a polynucleotide encoding a MYC polypeptide, a polynucleotide encoding a NFIB polypeptide and a polynucleotide encoding a LHX2 polypeptide, a polynucleotide encoding a NFIB polypeptide and a polynucleotide encoding a TFAP2A polypeptide, a polynucleotide encoding a NFIB polypeptide and a polynucleotide encoding a LHX2 polypeptide, a polynucleotide encoding a NFIB polypeptide and a polynucleotide encoding an IRX4 polypeptide, a polynucleotide encoding a NFIB polypeptide and a polynucleotide encoding a MYC polypeptide, a polynucleotide encoding a LHX2 polypeptide and a polynucleotide encoding a TFAP2A polypeptide, a polynucleotide encoding a LHX2 polypeptide and a polynucleotide encoding an IRX4 polypeptide, a polynucleotide encoding a LHX2 polypeptide and a polynucleotide encoding a MYC polypeptide, a polynucleotide encoding a TFAP2A polypeptide and a polynucleotide encoding an IRX4 polypeptide, a polynucleotide encoding a TFAP2A polypeptide and a polynucleotide encoding a MYC polypeptide, and a polynucleotide encoding an IRX4 polypeptide and a polynucleotide encoding a MYC polypeptide.

In some embodiments, a set of at least three agents is provided, e.g., a p53 agent, a NF-I agent, and a Lhx agent; a p53 agent, a NF-I agent, and a TFAP2 agent; a p53 agent, a NF-I agent, and an Irx agent; a p53 agent, a NF-I agent, and a Myc agent; a p53 agent, a Lhx agent, and a TFAP2 agent; a p53 agent, a Lhx agent, and an Irx agent; a p53 agent, a Lhx agent, and a Myc agent; a p53 agent, a TFAP2 agent, and an Irx agent; a p53 agent, a TFAP2 agent, and a Myc agent; a p53 agent, an Irx agent, and a Myc agent; a NF-I agent, a Lhx agent, and a TFAP2 agent; a NF-I agent, a Lhx agent, and an Irx agent; a NF-I agent, a Lhx agent, and Myc agent; a NF-I agent, a TFAP2 agent, and an Irx agent; a NF-I agent, a TFAP2 agent, and a Myc agent; a NF-I agent, an Irx agent, and a Myc agent; a Lhx agent, a TFAP2 agent, and an Irx agent; a Lhx agent, a TFAP2 agent, and a Myc agent; a Lhx agent, an Irx agent, and a Myc Agent; or a TFAP2 agent, an Irx agent, and a Myc agent. In some embodiments, a set of at least three agents is provided selected from the group consisting of a TP63 agent, a NF-I agent, and a Lhx2 agent; a TP63 agent, a NF-I agent, and a TFAP2A agent; a TP63 agent, a NF-I agent, and an IRX4 agent; a TP63 agent, a NF-I agent, and a MYC agent; a TP63 agent, a Lhx2 agent, and a TFAP2A agent; a TP63 agent, a Lhx2 agent, and an IRX4 agent; a TP63 agent, a Lhx2 agent, and a MYC agent; a TP63 agent, a TFAP2A agent, and an IRX4 agent; a TP63 agent, a TFAP2A agent, and a MYC agent; a TP63 agent, an IRX4 agent, and a MYC agent; a NF-I agent, a Lhx2 agent, and a TFAP2A agent; a NF-I agent, a Lhx2 agent, and an IRX4 agent; a NF-I agent, a Lhx2 agent, and MYC agent; a NF-I agent, a TFAP2A agent, and an IRX4 agent; a NF-I agent, a TFAP2A agent, and a MYC agent; a NF-I agent, an IRX4 agent, and a MYC agent; a Lhx2 agent, a TFAP2A agent, and an IRX4 agent; a Lhx2 agent, a TFAP2A agent, and a MYC agent; a Lhx2 agent, an IRX4 agent, and a MYC Agent; or a TFAP2A agent, an IRX4 agent, and a MYC agent. In some embodiments, a set of at least three agents is provided selected from the group consisting of a polynucleotide encoding a TP63 polypeptide, a polynucleotide encoding a NFIB polypeptide, and a polynucleotide encoding a Lhx2 polypeptide; a polynucleotide encoding a TP63 polypeptide, a polynucleotide encoding a NFIB polypeptide, and a polynucleotide encoding a TFAP2A polypeptide; a polynucleotide encoding a TP63 polypeptide, a polynucleotide encoding a NFIB polypeptide, and a polynucleotide encoding an IRX4 polypeptide; a polynucleotide encoding a TP63 polypeptide, a polynucleotide encoding a NFIB polypeptide, and a polynucleotide encoding a MYC polypeptide; a polynucleotide encoding a TP63 polypeptide, a polynucleotide encoding a Lhx2 polypeptide, and a polynucleotide encoding a TFAP2A polypeptide; a polynucleotide encoding a TP63 polypeptide, a polynucleotide encoding a Lhx2 polypeptide, and a polynucleotide encoding an IRX4 polypeptide; a polynucleotide encoding a TP63 polypeptide, a polynucleotide encoding a Lhx2 polypeptide, and a polynucleotide encoding a MYC polypeptide; a polynucleotide encoding a TP63 polypeptide, a polynucleotide encoding a TFAP2A polypeptide, and a polynucleotide encoding an IRX4 polypeptide; a polynucleotide encoding a TP63 polypeptide, a polynucleotide encoding a TFAP2A polypeptide, and a polynucleotide encoding a MYC polypeptide; a polynucleotide encoding a TP63 polypeptide, a polynucleotide encoding an IRX4 polypeptide, and a polynucleotide encoding a MYC polypeptide; a polynucleotide encoding a NFIB polypeptide, a polynucleotide encoding a Lhx2 polypeptide, and a polynucleotide encoding a TFAP2A polypeptide; a polynucleotide encoding a NFIB polypeptide, a polynucleotide encoding a Lhx2 polypeptide, and a polynucleotide encoding an IRX4 polypeptide; a polynucleotide encoding a NFIB polypeptide, a polynucleotide encoding a Lhx2 polypeptide, and a polynucleotide encoding a MYC polypeptide; a polynucleotide encoding a NFIB polypeptide, a polynucleotide encoding a TFAP2A polypeptide, and a polynucleotide encoding an IRX4 polypeptide; a polynucleotide encoding a NFIB polypeptide, a polynucleotide encoding a TFAP2A polypeptide, and a polynucleotide encoding a MYC polypeptide; a polynucleotide encoding a NFIB polypeptide, a polynucleotide encoding an IRX4 polypeptide, and a polynucleotide encoding a MYC polypeptide; a polynucleotide encoding a Lhx2 polypeptide, a polynucleotide encoding a TFAP2A polypeptide, and a polynucleotide encoding an IRX4 polypeptide; a polynucleotide encoding a Lhx2 polypeptide, a polynucleotide encoding a TFAP2A polypeptide, and a polynucleotide encoding a MYC polypeptide; a polynucleotide encoding a Lhx2 polypeptide, a polynucleotide encoding an IRX4 polypeptide, and a polynucleotide encoding a MYC polypeptide Agent; or a polynucleotide encoding a TFAP2A polypeptide, a polynucleotide encoding an IRX4 polypeptide, and a polynucleotide encoding a MYC polypeptide. For example, in some embodiments, a set of three agents is provided including a polynucleotide encoding LHX2, a polynucleotide encoding a TFAP2A polypeptide, and a polynucleotide encoding a MYC polypeptide.

In some embodiments, a set of at least four agents is provided, e.g., a p53 agent, a NF-I agent, a Lhx agent, and a TFAP2 agent; a p53 agent, a NF-I agent, a Lhx agent, and a Irx agent; a p53 agent, a NF-I agent, a Lhx agent, and a Myc agent; a p53 agent, a Lhx agent, a TFAP2 agent, and an Irx agent; a p53 agent, a Lhx agent, a TFAP2 agent, and a Myc agent; a p53 agent, a TFAP2 agent, an Irx agent, and a Myc agent; a p53 agent, a TRAF2 agent, a Irx agent, and a Myc agent; a NF-I agent, a Lhx agent, a TFAP2 agent, and an Irx agent; a NF-I agent, a Lhx agent, a TFAP2 agent, and a Myc agent; or a Lhx agent, a TFAP2 agent, an Irx agent, and a Myc agent. In some embodiments, a set of at least four agents is provided selected from the group consisting of a TP63 agent, a NFIB agent, a Lhx2 agent, and a TFAP2A agent; a TP63 agent, a NFIB agent, a Lhx2 agent, and a IRX4 agent; a TP63 agent, a NFIB agent, a Lhx2 agent, and a MYC agent; a TP63 agent, a Lhx2 agent, a TFAP2A agent, and an IRX4 agent; a TP63 agent, a Lhx2 agent, a TFAP2A agent, and a MYC agent; a TP63 agent, a TFAP2A agent, an IRX4 agent, and a MYC agent; a TP63 agent, a TRAF2 agent, a IRX4 agent, and a MYC agent; a NFIB agent, a Lhx2 agent, a TFAP2A agent, and an IRX4 agent; a NFIB agent, a Lhx2 agent, a TFAP2A agent, and a MYC agent; and a Lhx2 agent, a TFAP2A agent, an IRX4 agent, and a MYC agent. In some embodiments, a set of at least four agents is provided selected from the group consisting of a polynucleotide encoding a TP63 polypeptide, a polynucleotide encoding a NFIB polypeptide, a polynucleotide encoding a Lhx2 polypeptide, and a polynucleotide encoding a TFAP2A polypeptide; a polynucleotide encoding a TP63 polypeptide, a polynucleotide encoding a NFIB polypeptide, a polynucleotide encoding a Lhx2 polypeptide, and a polynucleotide encoding an IRX4 polypeptide; a polynucleotide encoding a TP63 polypeptide, a polynucleotide encoding a NFIB polypeptide, a polynucleotide encoding a Lhx2 polypeptide, and a polynucleotide encoding a MYC polypeptide; a polynucleotide encoding a TP63 polypeptide, a polynucleotide encoding a Lhx2 polypeptide, a polynucleotide encoding a TFAP2A polypeptide, and a polynucleotide encoding an IRX4 polypeptide; a polynucleotide encoding a TP63 polypeptide, a polynucleotide encoding a Lhx2 polypeptide, a polynucleotide encoding a TFAP2A polypeptide, and a polynucleotide encoding a MYC polypeptide; a polynucleotide encoding a TP63 polypeptide, a polynucleotide encoding a TFAP2A polypeptide, a polynucleotide encoding an IRX4 polypeptide, and a polynucleotide encoding a MYC polypeptide; a polynucleotide encoding a TP63 polypeptide, a polynucleotide encoding TRAF2, a polynucleotide encoding an IRX4 polypeptide, and a polynucleotide encoding a MYC polypeptide; a polynucleotide encoding a NFIB polypeptide, a polynucleotide encoding a Lhx2 polypeptide, a polynucleotide encoding a TFAP2A polypeptide, and a polynucleotide encoding an IRX4 polypeptide; a polynucleotide encoding a NFIB polypeptide, a polynucleotide encoding a Lhx2 polypeptide, a polynucleotide encoding a TFAP2A polypeptide, and a polynucleotide encoding a MYC polypeptide; and a polynucleotide encoding a Lhx2 polypeptide, a polynucleotide encoding a TFAP2A polypeptide, a polynucleotide encoding an IRX4 polypeptide, and a polynucleotide encoding a MYC polypeptide. For example, in some embodiments, a set of four agents is provided including a polynucleotide encoding a TP63 polypeptide, a polynucleotide encoding LHX2, a polynucleotide encoding a TFAP2A polypeptide, and a polynucleotide encoding a MYC polypeptide.

In some embodiments, a set of at least five agents is provided, e.g., a p53 agent, a NF-I agent, a Lhx agent, a TFAP2 agent, and an Irx agent; a p53 agent, a NF-I agent, a Lhx agent, a TFAP2 agent, and a Myc agent; a p53 agent, a Lhx agent, a TFAP2 agent, an Irx agent, and a Myc agent; a p53 agent, a NF-I agent, a TFAP2 agent, an Irx agent, and a Myc agent; a p53 agent, a NF-I agent, a Lhx agent, an Irx agent, and a Myc agent; a p53 agent, a NF-I agent, a Lhx agent, a TFAP2 agent, and a Myc agent; a NF-I agent, a Lhx agent, a TFAP2 agent, an Irx agent, and a Myc agent. In some embodiments, a set of at least five agents is provided selected from the group consisting of a TP63 agent, a NFIB agent, a LHX2 agent, a TFAP2A agent, and an IRX4 agent; a TP63 agent, a NFIB agent, a LHX2 agent, a TFAP2A agent, and a MYC agent; a TP63 agent, a LHX2 agent, a TFAP2A agent, an IRX4 agent, and a MYC agent; a TP63 agent, a NFIB agent, a TFAP2A agent, an IRX4 agent, and a MYC agent; a TP63 agent, a NFIB agent, a LHX2 agent, an IRX4 agent, and a MYC agent; a TP63 agent, a NFIB agent, a LHX2 agent, a TFAP2A agent, and a MYC agent; a NFIB agent, a LHX2 agent, a TFAP2A agent, an IRX4 agent, and a MYC agent. In some embodiments, a set of at least five agents is provided selected from the group consisting of a polynucleotide encoding a TP63 polypeptide, a polynucleotide encoding a NFIB polypeptide, a polynucleotide encoding a LHX2 polypeptide, a polynucleotide encoding a TFAP2A polypeptide, and a polynucleotide encoding an IRX4 polypeptide; a polynucleotide encoding a TP63 polypeptide, a polynucleotide encoding a NFIB polypeptide, a polynucleotide encoding a LHX2 polypeptide, a polynucleotide encoding a TFAP2A polypeptide, and a polynucleotide encoding a MYC polypeptide; a polynucleotide encoding a TP63 polypeptide, a polynucleotide encoding a LHX2 polypeptide, a polynucleotide encoding a TFAP2A polypeptide, a polynucleotide encoding an IRX4 polypeptide, and a polynucleotide encoding a MYC polypeptide; a polynucleotide encoding a TP63 polypeptide, a polynucleotide encoding a NFIB polypeptide, a polynucleotide encoding a TFAP2A polypeptide, a polynucleotide encoding an IRX4 polypeptide, and a polynucleotide encoding a MYC polypeptide; a polynucleotide encoding a TP63 polypeptide, a polynucleotide encoding a NFIB polypeptide, a polynucleotide encoding a LHX2 polypeptide, a polynucleotide encoding an IRX4 polypeptide, and a polynucleotide encoding a MYC polypeptide; a polynucleotide encoding a TP63 polypeptide, a polynucleotide encoding a NFIB polypeptide, a polynucleotide encoding a LHX2 polypeptide, a polynucleotide encoding a TFAP2A polypeptide, and a polynucleotide encoding a MYC polypeptide; a polynucleotide encoding a NFIB polypeptide, a polynucleotide encoding a LHX2 polypeptide, a polynucleotide encoding a TFAP2A polypeptide, a polynucleotide encoding an IRX4 polypeptide, and a polynucleotide encoding a MYC polypeptide.

In some embodiments, a set of at least six agents is provided, e.g., a p53 agent, a NF-I agent, a Lhx agent, a TFAP2 agent, an Irx agent, and a Myc agent. In some embodiments, a set of at least six agents is provided including a TP63 agent, a NFIB agent, a LHX2 agent, a TFAP2A agent, an IRX4 agent, and a MYC agent. In some embodiments, a set of at least six agents is provided including a polynucleotide encoding a TP63 polypeptide, a polynucleotide encoding a NFIB polypeptide, a polynucleotide encoding a LHX2 polypeptide, a polynucleotide encoding a TFAP2A polypeptide, a polynucleotide encoding an IRX4 polypeptide, and a polynucleotide encoding a MYC polypeptide.

All GenBank Accession No. sequences disclosed herein existed in the National Institutes of Health (NIH) genetic sequence database (GenBank) as at the date of filing of this priority application.

### Cell Culture

Cells contacted in vitro with the HFSCR system of reagents may be incubated in the presence of the reagent(s) for about 1 hour to about 8 weeks, e.g., 1 hour to 8 weeks, 2 hours to 8 weeks, 4 hours to 8 weeks, 6 hours to 8 weeks, 12 hours to 8 weeks, 18 hours to 8 weeks, 24 hours to 8 weeks, 48 hours to 8 weeks, 72 hours to 8 weeks, 96 hours to 8 weeks, 1 week to 8 weeks, 2 weeks to 8 weeks, 4 weeks to 8 weeks, 1 hour to 6 weeks, 2 hours to 6 weeks, 4 hours to 6 weeks, 6 hours to 6 weeks, 12 hours to 6 weeks, 18 hours to 6 weeks, 24 hours to 6 weeks, 48 hours to 6 weeks, 72 hours to 6 weeks, 96 hours to 6 weeks, 1 week to 6 weeks, 2 weeks to 6 weeks, 4 weeks to 6 weeks, 1 hour to 4 weeks, 2 hours to 4 weeks, 4 hours to 4 weeks, 6 hours to 4 weeks, 12 hours to 4 weeks, 18 hours to 4 weeks, 24 hours to 4 weeks, 48 hours to 4 weeks, 72 hours to 4 weeks, 96 hours to 4 weeks, 1 week to 4 weeks, 2 weeks to 4 weeks, 1 hour to 2 weeks, 2 hours to 2 weeks, 4 hours to 2 weeks, 6 hours to 2 weeks, 12 hours to 2 weeks, 18 hours to 2 weeks, 24 hours to 2 weeks, 48 hours to 2 weeks, 72 hours to 2 weeks, 96 hours to 2 weeks, or 1 week to 2 weeks. For example, in some embodiments, a population of non-HFS cells is incubated in the presence of the HFSCR system for a period of 1 hour to 6 weeks. In some embodiments, replacement of the HFSCR system may be replaced with a frequency of about every day to about every 14 days, e.g., every day, 1.5 days, 2 days, 3 days, 4 days, 5 days, 6 days, 7 days, or 14 days. The reagent(s) may be provided to the subject cells one or more times, e.g., one time, twice, three times, or more than three times, and the cells allowed to incubate with the reagent(s) for some amount of time following each contacting event, e.g., 24 hours to 6 weeks, after which time the media is replaced with fresh media and the cells are cultured further.

After contacting the non-HFS cells with the HFSCR system, the contacted cells may be cultured so as to promote the survival of HFSCs, HFPCs, or differentiated hair follicles. Methods and reagents for culturing cells, HFSCs, HFPCs, and differentiated hair follicles and for isolating HFSCs, HFPCs, and differentiated hair follicles are well known in the art, any of which may be used in the present invention to grow and isolate the iHFSCs and/or differentiated progeny.

For example, the non-HFS cells (either pre- or post-contacting with the HFSCR systems) may be plated on Matrigel or a similar basement membrane extract, a synthetic hydrogel, gelatin, collagen, laminin or other substrate as known in the art. The cells may be cultured in media such as DMEM, supplemented with factors. Alternatively, the contacted cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by use of growth factors and/or stromal cells associated with HFSC survival and differentiation.

In some embodiments a population of non-auditory iHFSCs are dissociated to form a cell suspension. Cell dissociation methods are well known in the art and can include enzymatic dissociation (e.g. trypsin dissociation), chemical dissociation (e.g., EDTA or EGTA dissociation), or mechanical dissociation. In some embodiments, a population of dermal cells is added to the dissociated population of non-auditory iHFSCs. For example, in some embodiments, the population of dermal cells are fibroblasts, smooth muscle cells, connective tissue cells, dermal papilla cells, and adipocytes.

In some embodiments, non-auditory iHFSCs are cultured on a scaffold. For example, a scaffold can include, but is not limited to, a plastic, a Matrigel or similar basement membrane extract, gelatin, collagen, or laminin matrix. In some embodiments, non-auditory iHFSCs are cultured on the scaffold for 30 minutes to 48 hours, e.g., 30 minutes to 24 hours, 30 minutes to 12 hours, 30 minutes to 6 hours, 30 minutes to 3 hours, 30 minutes to 2 hours, 30 minutes to 1 hour, 1 hour to 48 hours, 1 hour to 24 hours, 1 hour to 12 hours, 1 hour to 6 hours, 1 hour to 3 hours, or 1 hour to 2 hours. In some embodiments, non-auditory iHFSCs are cultured in combination with dermal cells on the scaffold for 30 minutes to 48 hours, e.g., 30 minutes to 24 hours, 30 minutes to 12 hours, 30 minutes to 6 hours, 30 minutes to 3 hours, 30 minutes to 2 hours, 30 minutes to 1 hour, 1 hour to 48 hours, 1 hour to 24 hours, 1 hour to 12 hours, 1 hour to 6 hours, 1 hour to 3 hours, or 1 hour to 2 hours.

### Methods of Treatment

iHFSCs or differentiated progeny cells produced by the above in vitro methods may be used in cell replacement or cell transplantation therapy to treat diseases. Specifically, iHFSCs and/or differentiated progeny may be transferred to subjects suffering from a wide range of diseases, conditions, or disorders with a hair loss component, i.e. with hair loss symptoms, for example to reconstitute or supplement hair forming units in a recipient. The therapy may be directed at treating the cause of the disease; or alternatively, the therapy may be to treat the effects of the disease or condition. For example, the therapy may be directed at replacing hair follicles whose death or senescence caused the disease.

The iHFSCs and/or differentiated progeny cells may be transferred to, or close to, an injured site in a subject; or the cells can be introduced to the subject in a manner allowing the cells to migrate, or home, to the injured site. The transferred cells may advantageously replace the damaged or injured cells and allow improvement in the overall condition of the subject. In some instances, the transferred cells may stimulate tissue regeneration or repair.

In some cases, the iHFSCs and/or differentiated progeny cells or a sub-population of iHFSCs and/or differentiated progeny cells may be purified or isolated from the rest of the cell culture prior to transferring to the subject. In other words, one or more steps may be executed to enrich for the iHFSCs and/or differentiated progeny cells or a subpopulation of iHFSCs or differentiated progeny cells, i.e. to provide an enriched population of iHFSCs or differentiated progeny cells or subpopulation of iHFSCs or differentiated progeny cells. In some cases, one or more antibodies specific for a marker of cells of the HFSCs or differentiated progeny lineages or a marker of a sub-population of cells of HFSCs or differentiated progeny lineages are incubated with the cell population and those bound cells are isolated. In other cases, the iHFSCs or differentiated progeny cells or a sub-population of the iHFSCs or differentiated progeny cells express a marker that is a reporter gene, e.g., EGFP, dsRED, lacz, and the like, that is under the control of a HFSC-specific, HFPC-specific, or differentiated hair cell-specific promoter which is then used to purify or isolate the iHFSCs or differentiated progeny cells or a subpopulation thereof.

By a marker it is meant that, in cultures comprising non-HFS cells that have been reprogrammed to become iHFSCs, the marker is expressed by the cells of the culture that will develop, are developing, and/or have developed into hair follicles. It will be understood by those of skill in the art that the stated expression levels reflect detectable amounts of the marker protein on or in the cell. A cell that is negative for staining (the level of binding of a marker-specific reagent is not detectably different from an isotype matched control) may still express minor amounts of the marker. And while it is commonplace in the art to refer to cells as “positive” or “negative” for a particular marker, actual expression levels are a quantitative trait. The number of molecules on the cell surface can vary by several logs, yet still be characterized as “positive”.

Cells of interest, i.e. cells expressing the marker of choice, may be enriched for, that is, separated from the rest of the cell population, by a number of methods that are well known in the art. For example, flow cytometry, e.g., fluorescence activated cell sorting (FACS), may be used to separate the cell population based on the intrinsic fluorescence of the marker, or the binding of the marker to a specific fluorescent reagent, e.g., a fluorophore-conjugated antibody, as well as other parameters such as cell size and light scatter. In other words, selection of the cells may be affected by flow cytometry. Although the absolute level of staining may differ with a particular fluorochrome and reagent preparation, the data can be normalized to a control. To normalize the distribution to a control, each cell is recorded as a data point having a particular intensity of staining. These data points may be displayed according to a log scale, where the unit of measure is arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. The “low” positively stained cells have a level of staining above the brightness of an isotype matched control, but are not as intense as the most brightly staining cells normally found in the population. An alternative control may utilize a substrate having a defined density of marker on its surface, for example a fabricated bead or cell line, which provides the positive control for intensity.

Other methods of separation, i.e. methods by which selection of cells may be affected, based upon markers include, for example, magnetic activated cell sorting (MACS), immunopanning, and laser capture microdissection.

Enrichment of iHFSC populations or a subpopulation of iHFSCs or differentiated progeny cells may be performed about 3 days or more after contacting the non-HFS cells with the HFSCR system, e.g., 3 days, 4 days, 5 days, 6 days, 7 days, 10 days, 14 days, 21 days, 30 days or 40 days after contacting the non-HFS cells with the HFSCR system. Populations that are enriched by selecting for the expression of one or more markers will usually have at least about 80% cells of the selected phenotype, more usually at least 90% cells and may be 95% of the cells, or more, of the selected phenotype. Subjects in need of hair transplantation therapy, e.g., a subject suffering from a condition associated with the loss of hair or with aberrantly functioning hair follicles, could especially benefit from therapies that utilize cells derived by the methods of the invention. Examples of such diseases, disorders and conditions include disorders which affect hair growth including androgenic alopecia, alopecia areata, telogen effluvium, trichotillomania, traction alopecia, tinea capitis, cicatricial alopecia. Examples of other conditions which may affect hair growth include scarring and trauma to the skin including chemical or heat induced burns. In some approaches, the reprogrammed non-HFS cells, i.e. iHFSCs or differentiated progeny may be transplanted directly to an injured site to treat a hair loss condition or in combination with such techniques known in the art such as follicular unit transplantation, follicular unit strip transplantation, and the like. In other approaches, the cells derived by the methods of the invention are engineered to respond to cues that can target their migration into existing epidermis, hair follicles, dermis or components of the skin. The iHFSCs may be administered in any physiologically acceptable medium. iHFSCs may be provided prior to differentiation, i.e. they may be provided in an undifferentiated state and allowed to differentiate in vivo, or they may be allowed to differentiate for a period of time ex vivo and provided following differentiation. iHFSCs may be cultured and/or differentiated prior to administration and they can be cultured and/or differentiated together as a co-culture prior to administration e.g., to support their growth and/or organization in the tissue to which they are being transplanted. iHFSCs may be provided alone or with a suitable substrate, matrix (e.g., plastic, Matrigel or similar basement membrane extract, gelatin, collagen, or laminin matrix), in combination, or in combination with cells such as dermal cells e.g., to support their growth and/or organization in the tissue to which they are being transplanted. For example, in some embodiments dermal cells are fibroblasts, smooth muscle cells, connective tissue cells, dermal papilla cells, or adipocytes. In some embodiments, at least 1×10⁵ cells, at least 1×10⁶ cells, or at least 1×10⁷ cells are administered to a subject. The cells may be introduced to the subjects by topical surgery, injection, or the like. Examples of methods for local delivery include follicular unit transplantation, follicular unit strip therapy, or by implanting a device upon which the cells have been reversibly affixed. For example, in some embodiments, a scaffold supporting at least 100,000 non-auditory iHFSCs, at least 1×10⁶ non-auditory iHFSCs, or at least 1×10⁷ non-auditory iHFSCs is grafted onto the subject. In some embodiments, a scaffold supporting at least 100,000 non-auditory iHFSCs and dermal cells, at least 1×10⁶ non-auditory iHFSCs and dermal cells, or at least 1×10⁷ non-auditory iHFSCs and dermal cells is grafted onto the subject.

The number of administrations of treatment to a subject may vary. Introducing the iHFSCs and/or differentiated progeny cells into the subject may be a one-time event; but in certain situations, such treatment may elicit improvement for a limited period of time and require an on-going series of repeated treatments. In other situations, multiple administrations of the iHFSCs and/or differentiated progeny cells may be required before an effect is observed. The exact protocols depend upon the disease or condition, the stage of the disease and parameters of the individual subject being treated.

### Experimental or Screening Uses

Additionally or alternatively, iHFSCs and/or differentiated progeny produced by the above in vitro methods may be used as a basic research or drug discovery tool, for example to evaluate the phenotype of a genetic disease, e.g., to better understand the etiology of the disease, to identify target proteins for therapeutic treatment, to identify candidate agents with disease-modifying activity, i.e. an activity in modulating the survival or function of HFSCs or differentiated progeny cells in a subject suffering from a hair loss disease or disorder, e.g., to identify an agent that will be efficacious in treating the subject. For example, a candidate agent may be added to a cell culture comprising iHFSC and/or differentiated progeny cells derived from the subject's somatic cells, and the effect of the candidate agent assessed by monitoring output parameters such as iHFSCs or differentiated progeny cell survival, the ability of the iHFSCs or differentiated progeny cells to form hair or promote hair growth, the extent to which the iHFSCs and/or differentiated progeny cells continuously produce hair, and the like, by methods described herein and in the art.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g., mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to one or a plurality of cell samples, usually in conjunction with cells lacking the agent. The change in parameters in response to the agent is measured, and the result evaluated by comparison to reference cultures, e.g., in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e. any elements developed that perform the same function, regardless of structure. The scope of the present invention, therefore, is not intended to be limited to the exemplary embodiments shown and described herein. Rather, the scope and spirit of the present invention is embodied by the appended claims.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: Generation and Validation of Human Induced Hair Follicle Stem Cells

Induced hair follicle stem cells (iHFSCs) were produced by transducing polynucleotides encoding TP63, NFIB, LHX2, TFAP2A, IRX4 and MYC in lentiviral vectors containing tetracycline-inducible promoters into human fibroblasts (HFs purchased from Cell Applications) in human fibroblast growth medium. HFs were incubated with these viral particles for 12-24 hours to facilitate transduction. After this period, medium was replaced with fresh human fibroblast growth medium supplemented with tetracycline. 24-36 hours after, culture medium was replaced with hair follicle stem cell (HFSC) growth medium supplemented with tetracycline. Cells were cultured further for another 1-4 weeks in HFSC growth medium supplemented with tetracycline with the culture medium being replaced every 1-3 days. Cultures were passaged as a mixed population of cells upon confluence. During this 1-4 week period, greater than 0.1% of transdifferentiating cells acquired an epithelial cell like morphology and were termed putative iHFSCs. iHFSCs continued to grow in HFSC growth medium and were passaged or frozen in cell freezing medium upon reaching confluence.

Quantitative polymerase chain reaction (qPCR) analysis was used to measure expression of known HFSC markers (ECAD, KRT15, KLF5, HR, SOX9, TFAP2A, ITGA6, CTIP2, ESRP1, and TBX1) normalized to the house-keeping gene actin. To conduct qPCR analysis, mRNA was isolated from iHFSCs and negative control HF cells with Qiagen RNeasy kits according to manufacturer instructions. cDNA was produced with BioRad iScript Reverse Transcription Mix according to manufacturer instructions. qPCR was conducted on a BioRad C1000™ instrument with BioRad SsoAdvanced Universal SYBR® Green SuperMix according manufacturer instructions and with the following amplification protocol: 95° C. for 30 seconds followed by 40 cycles of [95° C. for 15 seconds, 60° C. for 30 seconds]. Primers nucleotide sequences used for qPCR assays are listed in TABLE 1 below.

TABLE 1
PRIMERS FOR qPCR ANALYSIS OF HFSC MARKER GENES

| Gene | Sense primer | Antisense primer |
|---|---|---|
| Actin | ccaaccgcgagaagatga | ccagaggcgtacagggatag |
| | (SEQ ID NO: 6) | (SEQ ID NO: 7) |
| ECAD | ggccaggaaatcacatccta | ggcagtgtctctccaaatcc |
| | (SEQ ID NO: 8) | (SEQ ID NO: 9) |
| KRT15 | agatgctgcttgacataaagacac | gctaccaccacctcctgaag |
| | (SEQ ID NO: 10) | (SEQ ID NO: 11) |
| KLF5 | ggctttactcaagcagatctcatc | cccttacccatgttgagacg |
| | (SEQ ID NO: 12) | (SEQ ID NO: 13) |
| HR | ggacagcatgatgagcagaa | caggcatggtatgtcctgaa |
| | (SEQ ID NO: 14) | (SEQ ID NO: 15) |
| SOX9 | aaacaccttgagccttaaaacg | aggcaggaggaaatgcacta |
| | (SEQ ID NO: 16) | (SEQ ID NO: 17) |
| TFAP2A | tgggaccacctggtattctg | ccagcacaactcaatacaatgc |
| | (SEQ ID NO: 18) | (SEQ ID NO: 19) |
| ITGA6 | agcctcttcggcttctcg | ttggctctctgcagtggaa |
| | (SEQ ID NO: 20) | (SEQ ID NO: 21) |
| CTIP2 | ctgggagagcaagtgttgg | ggaacatacaaccagggaccta |
| | (SEQ ID NO: 22) | (SEQ ID NO: 23) |
| ESRP1 | cccaaagaatgggtttgtattt | tggaggtttcaagatcaccat |
| | (SEQ ID NO: 24) | (SEQ ID NO: 25) |
| TBX1 | gtgccggtggacgataag | gagtccgggtggtagtgc |
| | (SEQ ID NO: 26) | (SEQ ID NO: 27) |

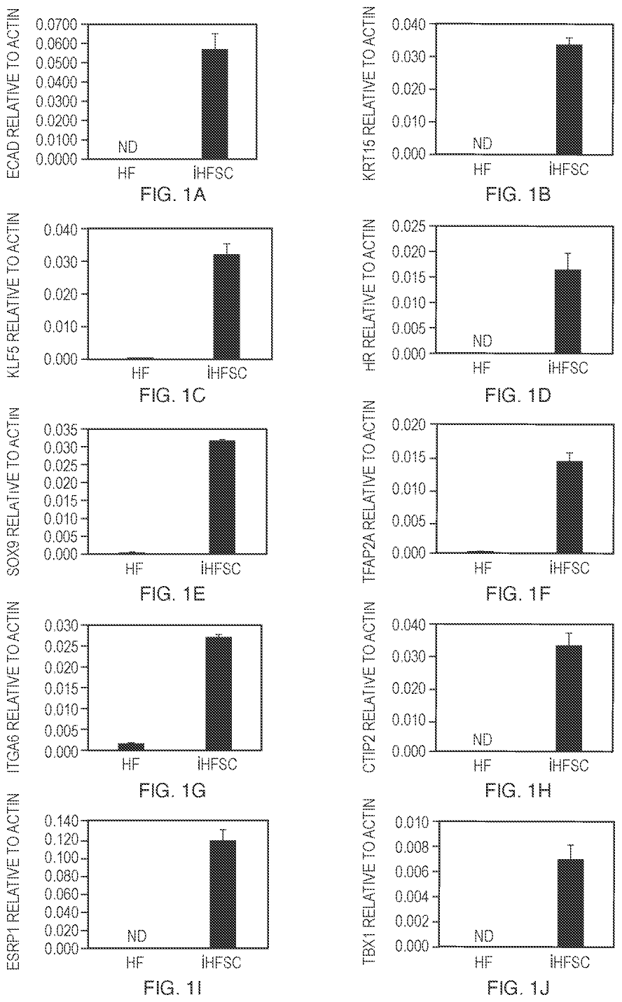

As shown in FIGS. 1A-J, transduced cells show increased expression of the hair stem cell marker genes ECAD (FIG. 1A), KRT15 (FIG. 1B), KLF5 (FIG. 1C), HR (FIG. 1D), SOX9 (FIG. 1E), TFAP2A (FIG. 1F), ITGA6 (FIG. 1G), CTIP2 (FIG. 1H), ESRP1 (FIG. 1I), and TBX1 (FIG. 1J) compared to HFs, indicating their transdifferentiation to iHFSCs. Crossover threshold (CT) cutoff set to 30 cycles. ND represents values that were Not Detected or detected above the CT cutoff of 30 cycles.

iHFSCs were also produced by transducing polynucleotides encoding TP63, NFIB, LHX2, TFAP2A, IRX4 and MYC or TP63, LHX2, TFAP2A, and MYC in lentiviral vectors containing tetracycline-inducible promoters into human epithelial cells (EPIs) in epithelial cell growth medium. EPIs were incubated with these viral particles for 12-24 hours to facilitate transduction. After this period, medium was replaced with fresh human epithelial cell growth medium supplemented with tetracycline. 24-36 hours after, culture medium was replaced with hair follicle stem cell (HFSC) growth medium supplemented with tetracycline. Cells were cultured further for another 1-4 weeks in HFSC growth medium supplemented with tetracycline with the culture medium being replaced every 1-3 days. Cultures were passaged as a mixed population of cells upon confluence. During this 1-4 week period, greater than 0.1% of transdifferentiating cells acquired hair follicle stem cell like morphology and were termed putative iHFSCs. iHFSCs continued to grow in HFSC growth medium and were passaged or frozen in cell freezing medium upon reaching confluence.

Quantitative polymerase chain reaction (qPCR) analysis was used to measure expression of known HFSC markers (ECAD, KRT15, KLF5, HR, SOX9, TFAP2A, ITGA6, CTIP2, ESRP1, and TBX1) normalized to the house-keeping gene actin. To conduct qPCR analysis, mRNA was isolated from iHFSCs and negative control EPI cells with Qiagen RNeasy kits according to manufacturer instructions. cDNA was produced with BioRad iScript Reverse Transcription Mix according to manufacturer instructions. qPCR was conducted on a BioRad C1000™ instrument with BioRad SsoAdvanced Universal SYBR® Green SuperMix according manufacturer instructions and with the following amplification protocol: 95° C. for 30 seconds followed by 40 cycles of [95° C. for 15 seconds, 60° C. for 30) seconds]. Primer nucleotide sequences used for qPCR assays are listed in TABLE 1.

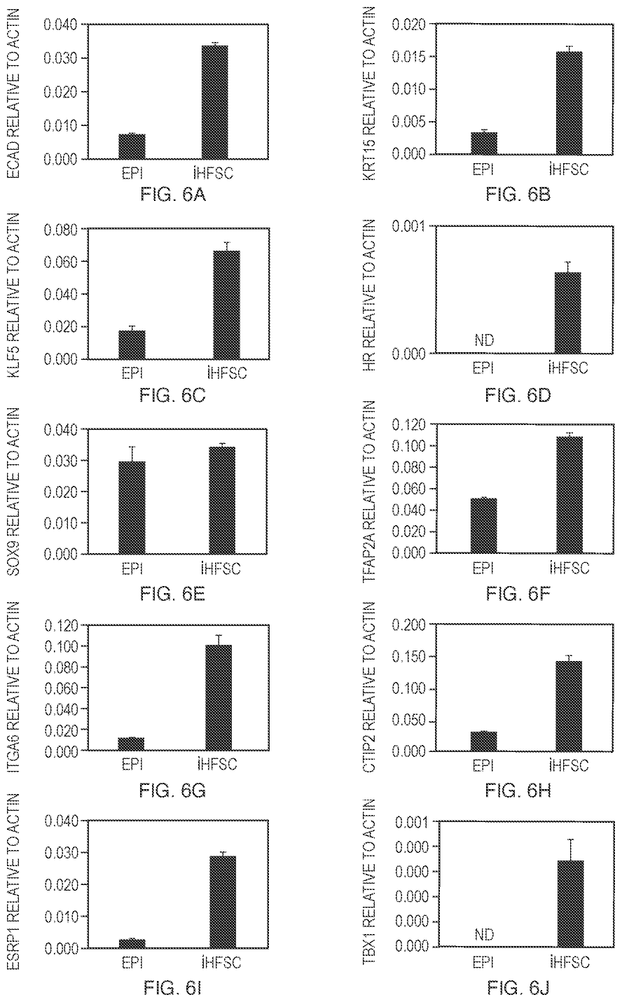

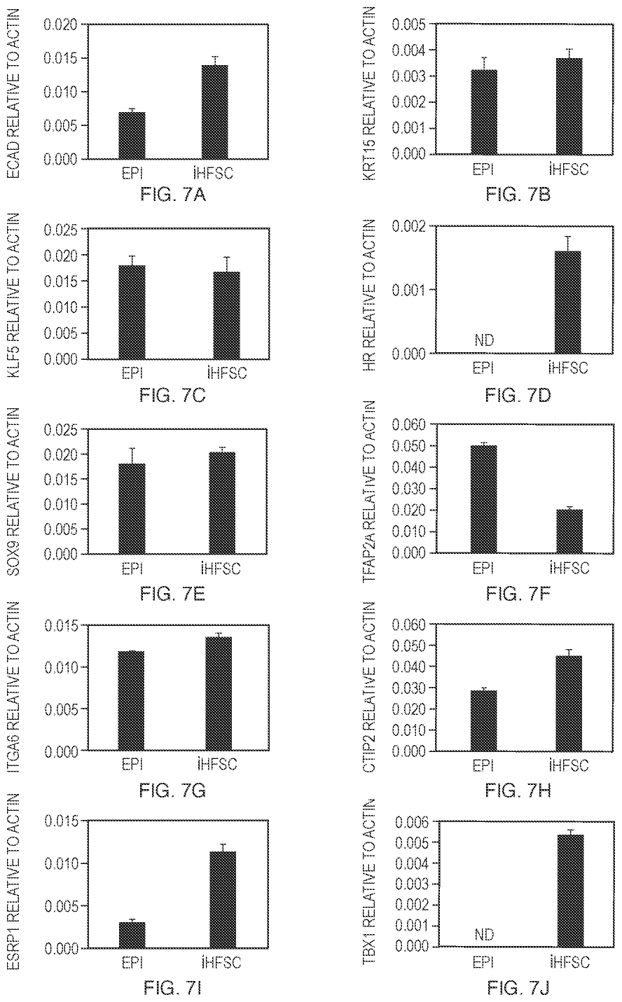

As shown in FIGS. 6A-J and FIG. 7A-J, transduced cells show sustained or increased expression of the hair stem cell marker genes ECAD (FIG. 6A, FIG. 7A), KRT15 (FIG. 6B, FIG. 7B), KLF5 (FIG. 6C, FIG. 7C), HR (FIG. 6D, FIG. 7D), SOX9 (FIG. 6E, FIG. 7E), TFAP2A (FIG. 6F, FIG. 7F), ITGA6 (FIG. 6G, FIG. 7G), CTIP2 (FIG. 6H, FIG. 7H), ESRP1 (FIG. 6I. FIG. 7I), and TBX1 (FIG. 6J, FIG. 7J) compared to EPIs, indicating their transdifferentiation to iHFSCs. Crossover threshold (CT) cutoff set to 30 cycles. ND represents values that were not detected or detected above the CT cutoff of 30 cycles.

iHFSCs were also produced by transducing polynucleotides encoding TP63, LHX2, TFAP2A, and MYC or LHX2, TFAP2A, and MYC in lentiviral vectors containing tetracycline-inducible promoters into human fibroblasts (HFs) in human fibroblast growth medium. HFs were incubated with these viral particles for 12-24 hours to facilitate transduction. After this period, medium was replaced with fresh human fibroblast growth medium supplemented with tetracycline. 24-36 hours after, culture medium was replaced with hair follicle stem cell (HFSC) growth medium supplemented with tetracycline. Cells were cultured further for another 1-4 weeks in HFSC growth medium supplemented with tetracycline with the culture medium being replaced every 1-3 days. Cultures were passaged as a mixed population of cells upon confluence. During this 1-4 week period, greater than 0.1% of transdifferentiating cells acquired hair follicle stem cell like morphology and were termed putative iHFSCs. iHFSCs continued to grow in HFSC growth medium and were passaged or frozen in cell freezing medium upon reaching confluence.

qPCR analysis was used to measure expression of known HFSC markers (ECAD, KRT15, KLF5, HR, SOX9, TFAP2A, ITGA6, CTIP2, ESRP1, and TBX1) normalized to the house-keeping gene actin. To conduct qPCR analysis, mRNA was isolated from iHFSCs and negative control HF cells with Qiagen RNeasy kits according to manufacturer instructions. cDNA was produced with BioRad iScript™ Reverse Transcription Mix according to manufacturer instructions. qPCR was conducted on a BioRad C1000™ instrument with BioRad SsoAdvanced Universal SYBRR Green SuperMix according manufacturer instructions and with the following amplification protocol: 95 C for 30 seconds followed by 40 cycles of [95° C. for 15 seconds, 60° C. for 30 seconds]. Primer nucleotide sequences used for qPCR assays are listed in TABLE 1.

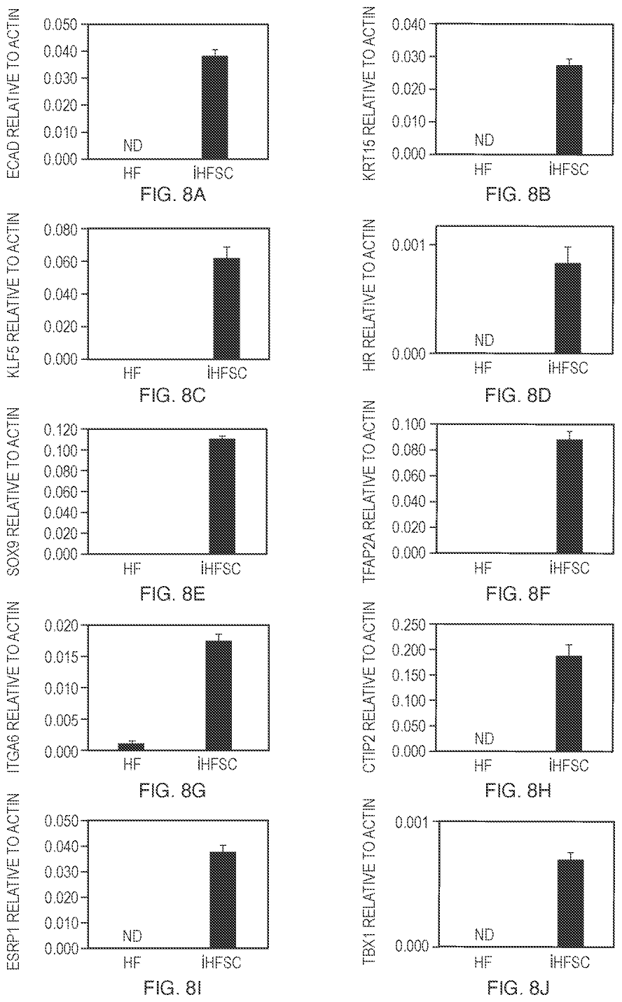

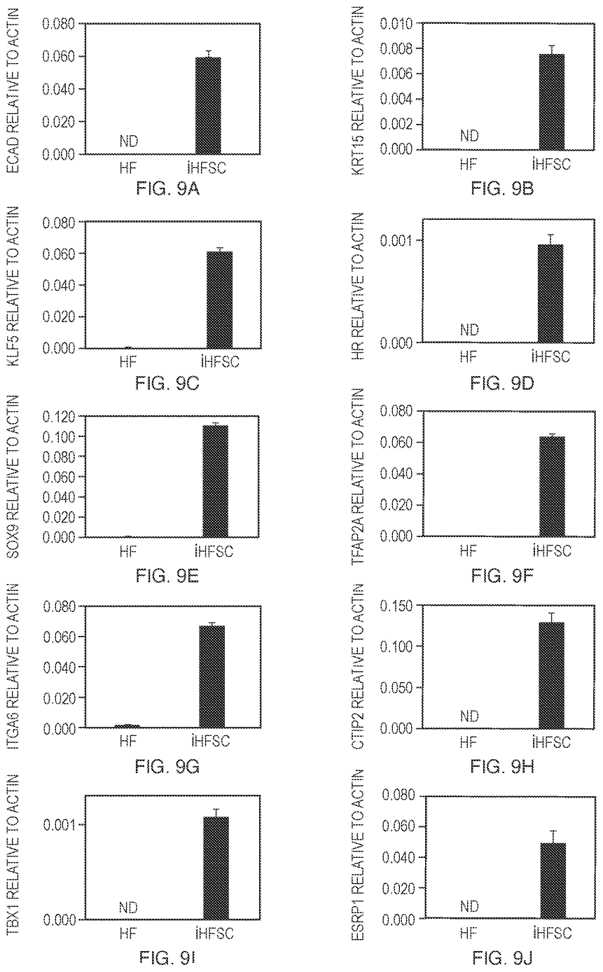

As shown in FIG. 8A-J and FIG. 9A-J, transduced cells show sustained or increased expression of the hair stem cell marker genes ECAD (FIG. 8A, FIG. 9A), KRT15 (FIG. 8B, FIG. 9B), KLF5 (FIG. 8C, FIG. 9C), HR (FIG. 8D, FIG. 9D), SOX9 (FIG. 8E, FIG. 9E), TFAP2A (FIG. 8F, FIG. 9F), ITGA6 (FIG. 8G, FIG. 9G), CTIP2 (FIG. 8H, FIG. 9H), ESRP1 (FIG. 8I. FIG. 9I), and TBX1 (FIG. 8J, FIG. 9J) compared to HFs, indicating their transdifferentiation to iHFSCs. Crossover threshold (CT) cutoff set to 30 cycles. ND represents values that were not detected or detected above the CT cutoff of 30 cycles.

Immunofluorescence microscopy was used to verify protein-level expression of HFSC markers in transduced cells. For immunofluorescence staining, cell cultures were fixed with 4% paraformaldehyde in phosphate buffered saline (PBS) for 20 minutes at room temperature, washed 3 times in PBS, and incubated in staining solution (5% CCS, 0.1% Triton-X 100 in PBS) for 20 minutes. Cultures were then incubated with antibody solutions comprising goat anti-ECAD (R&D Systems) diluted 1:20 in staining solution for 1 hour, Chicken anti-KRT15 (BioLegend) diluted 1:50 in staining solution, Rabbit anti-KLF5 (Abcam) diluted 1:200 in staining solution, or Rabbit anti-SOX9 (Invitrogen) diluted 1:50 in staining solution for 1 hour. Cells were then washed 3 times in PBS, incubated with their corresponding antibody chosen from Donkey IgG (H+L) anti-Goat Alexa Fluor™ 488 (Invitrogen), Donkey IgY (IgG) (H+L) anti-Chicken Alexa Fluor™ 488 (Jackson ImmunoResearch Laboratories, Inc.), Donkey IgG (H+L) anti-Rabbit Alexa Fluor™* 488 (Invitrogen), diluted 1:1000 in staining solution for 30 minutes. For ITGA6 staining, cells were incubated in staining solution (5% CCS in PBS) for 20 minutes. then for ITGA6 visualization, cells were incubated with an antibody solution of Rat anti-Human CD49f Fluorescein Isothiocyanate (FITC) (BD Pharmingen) diluted 1:200 in staining solution for 1 hour. All cultures were next washed 3 times in PBS, then incubated for 5 minutes in 4,6-diamidino-2-phenylindole (DAPI) (Sigma) diluted to 1 μg/ml in PBS, then rinsed and resuspended in PBS.

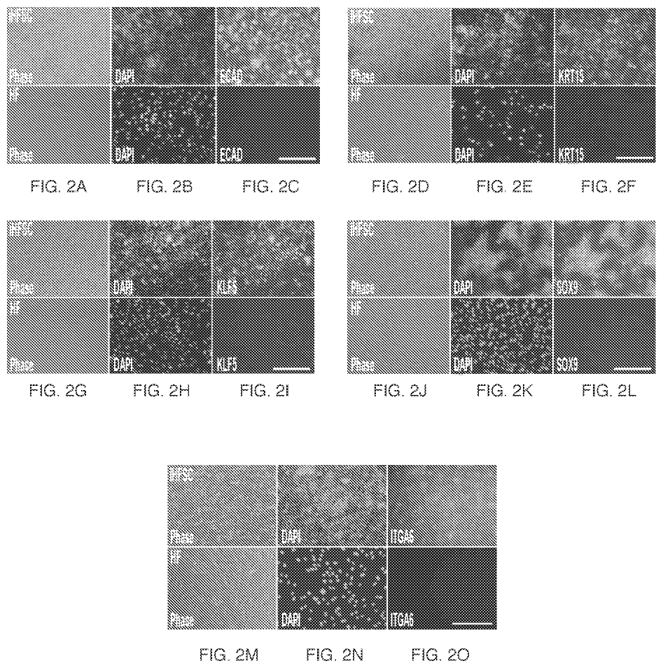

FIGS. 2A-2O show microscopy images of transduced cells (top micrographs) or negative control HFs (bottom micrographs). FIGS. 2A, 2D, 2G, 2J, and 2M show 20× phase contrast images, FIGS. 2B, 2E, 2H, 2K, and 2N show DAPI staining for nuclei visualization, and FIGS. 2C, 2F, 2I, 2L, and 2O show staining for ECAD, KRT15, KLF5, SOX9, and ITGA6, respectively, as visualized with Alexa Fluor™ 488 or FITC. Scale bar represents 200 μm. For all makers, staining is prominent in transduced cells and not visible in control cells, indicating that transduced cells were transdifferentiated into iHFSCs.

### Example 2: Isolation of Human iHFSCS

Fluorescent Activated Cell Sorting (FACS) was used on iHFSCs produced using the protocol described in EXAMPLE 1. Cultures if iHF were dissociated in TrypLE (ThermoFisher Scientific) for 5 minutes, washed in phosphate buffered saline (PBS), centrifuged at 300×g for 5 minutes and resuspended in staining solution (0.5% BSA in PBS). Cells were filtered through a 40 μm cell strainer and centrifuged for 5 minutes at 300 g prior to staining. For staining, cells were incubated for 30 minutes with Chicken anti-KRT15 (BioLegend) and Goat anti-ECAD (R&D Systems) with both diluted 1:25 in staining solution on ice. Cells were then washed with staining solution, centrifuged at 300×g for 5 minutes, washed with staining solution, centrifuged at 300×g for 5 minutes, then incubated for 30 minutes with Donkey IgY (IgG) (H+L) anti-Chicken Alexa Fluor™ 488 (Jackson™ 647 ImmunoResearch Laboratories, Inc.) and Donkey IgG (H+L) anti-Goat Alexa Fluor™ (Invitrogen) with both diluted at 1:1000 in staining solution on ice. Cells were then washed in staining solution, centrifuged at 300×g for 5 minutes, washed again with staining solution and centrifuged at 300×g for 5 minutes, then resuspended in staining solution supplemented with EDTA and Propidium Iodide (Invitrogen) according to the manufacturer's instructions. Stained samples were analyzed with an Attune NxT Flow Cytometer according to the manufacturer's instructions.

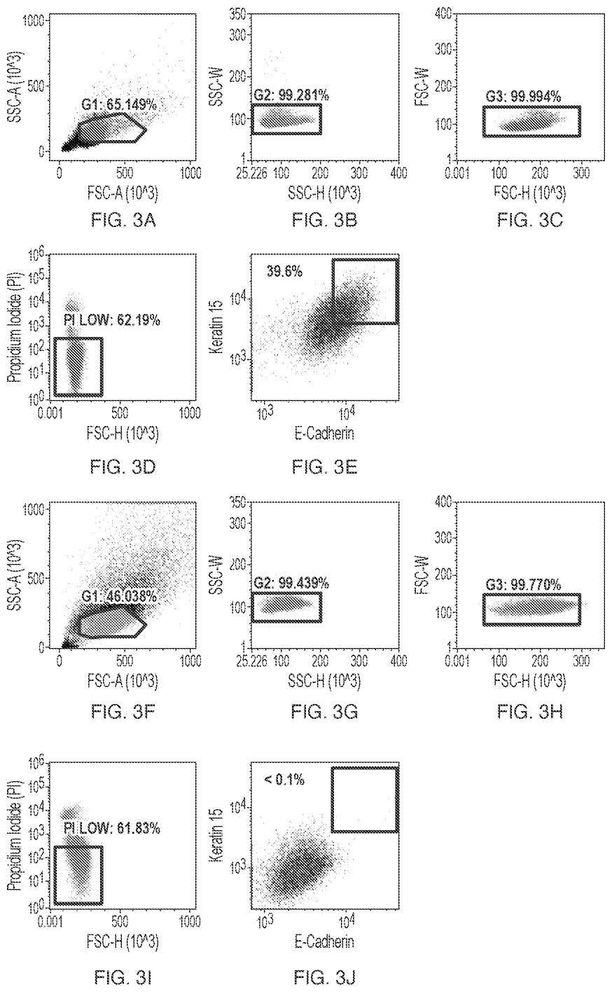

Flow cytometry analysis for iHFSCs are shown in FIGS. 3A-3E while negative control HFs are shown in FIGS. 3F-3J. Gating parameters for cell size are shown in FIGS. 3A-3C and FIGS. 3F-3H. Dead cells were excluded by Propidium Iodide (PI) staining as shown in FIG. 3D and FIG. 31. KRT15 and ECAD staining are shown in FIG. 3E and FIG. 3J. The boxes and accompanying numbers indicate the cell populations that are KRT15/ECAD double-positive, demonstrating the transdifferentiation of transduced cells into iHFSCs. Furthermore, these iHFSCs can be isolated and purified from a bulk population of cells by FACS based on KRT15/ECAD double positivity for further experimentation or usage.

### Example 3: Human iHFSCS have the Capacity to Self-Renew

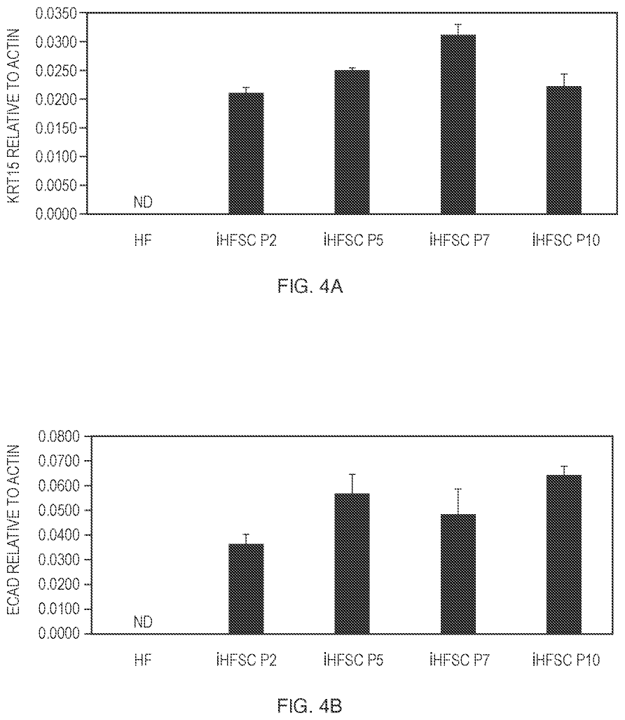

Maintenance of marker gene expression was used to analyze the ability of iHFSCs to self-renew: The protocol from EXAMPLE 1 was used to generate iHFSCs. Marker gene expression was assessed by qPCR analysis of mRNA isolated from iHFSCs at passage (P) 2, 5, 7, and 10, and negative control human fibroblast (HF) using the protocol from EXAMPLE 1. Primer nucleotide sequences used to detect actin. KRT15, and ECAD are listed in TABLE 1 above. CT cutoff set to 30 cycles. ND represents values that were Not Detected or detected above the CT cutoff of 30 cycles. Expression values of KRT15 (FIG. 4A) and ECAD (FIG. 4B) relative to Actin are shown for iHFSCs and negative control HFs. iHFSCs maintain upregulated levels of KRT15 and ECAD after multiple passages while both markers are not detected in negative control HFs, indicating that iHFSCs are capable of self-renewal.

### Example 4: Human iHFSCS Produce Human Hair in a Nude Mouse Model

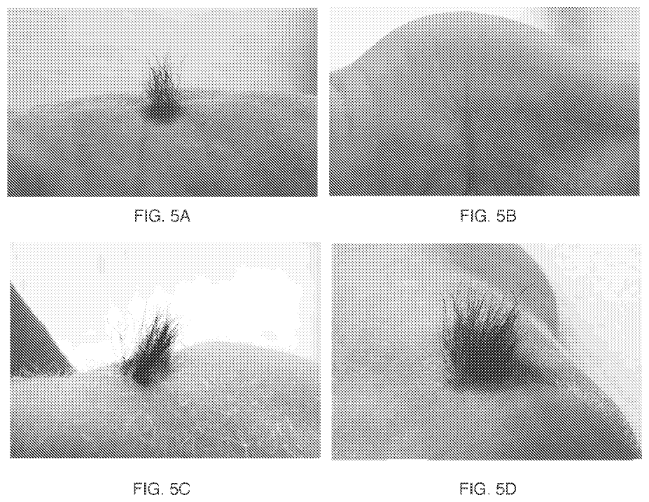

A nude mouse model was used to test the ability of grafted iHFSCs to produce hair. The protocol from EXAMPLE 1 was used to produce iHFSCs. For transplantation, iHFSCs were combined with mouse dermal cells isolated from C57BL/6 (Charles River) perinatal pups. To isolate dermal cells, dorsal skin was isolated, incubated in 1:1 Dispase:F12 or TrpLE overnight at 4° C. epidermis was manually removed from dermis and discarded, dermis was further incubated in 0.35% collagenase at 37° C. for 50 minutes, quenched with 10% FBS, then filtered through a 40 μm cell strainer. Cells were then counted and 2.5×10⁶ mouse dermal cells were combined with 5×10⁶ iHFSCs, pelleted at 300×g for 5 minutes, resuspended in DMEM:F12, plated on a plastic sheet and incubated at 37° C. for 1 hour. These molds were then grafted onto nude SCID mice (Charles River. Crl:NU (NCr)-Foxn1nu), which are deficient for hair growth, after excising the host skin at the graft site. Hair growth was assessed at 4 weeks post-transplantation (FIG. 5A) compared to time-matched negative controls (FIG. 5B). Hair growth was further assessed in separate experiment at 6 weeks and 9 weeks post-transplantation (FIGS. 5C and 5D, respectively). Hair growth resulting from the transplants could be differentiated from the short, white nude SCID mouse hair due to the black color and extended length of the iHFSC produced hair.

### Example 5: Grafting of Human iHFSCS onto Human Patients

iHFSCs are used to grow hair in human patients for the treatment of human hair loss conditions such as androgenic alopecia, alopecia areata, telogen effluvium, trichotillomania, traction alopecia, tinea capitis, and cicatricial alopecia. The method described in EXAMPLE 1 is used to generate iHFSCs from a patient's own cells, from an allogeneic donor, or from a cultured cell line. An appropriate number of cells, for example, but not limited to, 100,000, 1,000,000, or 10,000,000 iHFSCs is placed on a suitable support, such as a plastic sheet as described in EXAMPLE 4 or another suitable support comprised of Matrigel, a similar basement membrane extract, a synthetic hydrogel, or an extracellular membrane protein-based scaffold (e.g., collagen, fibronectin, gelatin, or laminin). iHFSCs optionally are combined with human dermal cells, or cells functionally equivalent to dermal cells, obtained from the patient, an allogeneic donor, or cell line. The support is grafted onto the patient at an appropriate site. The site of the graft is monitored in the short term (i.e. weeks to months) for the ability of the grafted iHFSCs to produce hair, as well as in the long term (i.e. years to decades) for the ability of the grafted iHFSCs to cause sustained hair growth. Adverse effects, such as skin irritation or other conditions that may arise from iHFSC grafting, are monitored to assess the safety of iHFSC grafting.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A composition comprising:
a population of non-hair follicle stem (HFS) cells;
a population of non-auditory induced hair follicle stem cells (iHFSCs) expressing one or more marker(s) selected from the group consisting of E-Cadherin (ECAD), keratin 15 (KRT15), Kruppel Like Factor 5 (KLF5), HR Lysine Demethylase and Nuclear Receptor Corepressor (HR), SRY-Box Transcription Factor 9 (SOX9), Transcription Factor AP-2 Alpha (TFAP2A), Integrin Subunit Alpha 6 (ITGA6), BAF Chromatin Remodeling Complex Subunit BCL11B (CTIP2), Epithelial Splicing Regulatory Protein 1 (ESRP1), and T-box Transcription Factor 1 (TBX1); and
a hair follicle stem cell reprogramming (HFSCR) system comprising one or more hair follicle stem cell reprogramming factor(s) selected from the group consisting of a p53 agent, a NF-I agent, an Lhx agent, a TFAP2 agent, an IRX agent, and a Myc agent,
wherein the HFSCR system causes transdifferentiation of one or more non-HFS cell into one or more non-auditory iHFSC.

2. The composition of claim 1, wherein the population of non-auditory iHFSCs have at least a two-fold increased expression of one or more marker(s), selected from the group consisting of ECAD, KRT15, KLF5, HR, SOX9, TFAP2A, ITGA6, CTIP2, ESRP1, and TBX1, as compared to the population of non-hair follicle stem cells.

3. The composition of claim 1, wherein the population of non-auditory iHFSCs have at least a five-fold increased expression of one or more marker(s) selected from the group consisting of ECAD, KRT15, KLF5, HR, SOX9, TFAP2A, ITGA6, CTIP2, ESRP1, and TBX1, as compared to the population of non-hair follicle stem cells.

4. The composition of claim 1, wherein the population of non-auditory iHFSCs have at least a ten-fold increased expression of one or more marker(s) as selected from the group consisting of ECAD, KRT15, KLF5, HR, SOX9, TFAP2A, ITGA6, CTIP2, ESRP1, and TBX1, as compared to the population of non-hair follicle stem cells.

5. (canceled)

6. (canceled)

7. (canceled)

8. The composition of claim 1, wherein the p53 agent is a p53 polypeptide, or functional fragment thereof, fused to a permeant domain.

9. The composition of claim 1, wherein the NF-I agent is an NF-I polypeptide, or functional fragment thereof, fused to a permeant domain.

10. The composition of claim 1, wherein the Lhx agent is an Lhx polypeptide, or functional fragment thereof, fused to a permeant domain.

11. The composition of claim 1, wherein the TFAP2 agent is a TFAP2 polypeptide, or functional fragment thereof, fused to a permeant domain.

12. The composition of claim 1, wherein the IRX agent is an IRX polypeptide, or functional fragment thereof, fused to a permeant domain.

13. The composition of claim 1, wherein the Myc agent is a Myc polypeptide, or functional fragment thereof, fused to a permeant domain.

14. The composition of claim 8, wherein the permeant domain comprises an amino acid sequence selected from the group consisting of:
RQIKIWFQNRRMKWKK (SEQ ID NO: 1), RKKRRQRRR (amino acids 49-57 of HIV-1 tat; SEQ ID NO:2), TRQARRNRRRRWRERQR (amino acids 34-50 of HIV-1 rev; SEQ ID NO:3), RRRRRRRRR (R9; SEQ ID NO:4), and RRRRRRRR (R8; SEQ ID NO:5).

15. The composition of claim 1, wherein the p53 agent is a nucleic acid encoding a p53 polypeptide, or functional fragment thereof.

16. The composition of claim 1, wherein the p53 agent is a polynucleotide encoding a TP63 polypeptide.

17. The composition of claim 1, wherein the NF-I agent is a nucleic acid encoding an NF-I polypeptide, or functional fragment thereof.

18. The composition of claim 1, wherein the NF-I agent is a polynucleotide encoding a NFIB polypeptide.

19. The composition of claim 1, wherein the Lhx agent is a nucleic acid encoding a Lhx polypeptide, or functional fragment thereof.

20. The composition of claim 1, wherein the Lhx agent is a polynucleotide encoding a LHX2 polypeptide.

21. The composition of claim 1, wherein the TFAP2 agent is a nucleic acid encoding a TFAP2 polypeptide, or functional fragment thereof.

22. The composition of claim 1, wherein the TFAP2 agent is a polynucleotide encoding a TFAP2A polypeptide.

23. The composition of claim 1, wherein the IRX agent is a nucleic acid encoding an IRX polypeptide, or functional fragment thereof.

24. The composition of any claim 1, wherein the IRX agent is a polynucleotide encoding an IRX4 polypeptide.

25. The composition of claim 1, wherein the Myc agent is a nucleic acid encoding a Myc polypeptide, or functional fragment thereof.

26. The composition of claim 1, wherein the Myc agent is a polynucleotide encoding a MYC polypeptide.

27. (canceled)

28. (canceled)

29. (canceled)

30. The composition of claim 1, wherein the HFSCR comprises at least three hair follicle stem cell reprogramming factors selected from the group consisting of: a nucleic acid encoding a p53 polypeptide, a nucleic acid encoding a NF-I polypeptide, a nucleic acid encoding a Lhx polypeptide, a nucleic acid encoding a TFAP2 polypeptide, a nucleic acid encoding an IRX polypeptide, and a nucleic acid encoding a Myc polypeptide.

31. The composition of claim 30, wherein the HFSCR comprises at least three hair follicle stem cell reprogramming factors selected from the group consisting of: a nucleic acid encoding a TP63 polypeptide, a nucleic acid encoding a NFIB polypeptide, a nucleic acid encoding a LHX2 polypeptide, a nucleic acid encoding a TFAP2A polypeptide, a nucleic acid encoding an IRX4 polypeptide, and a nucleic acid encoding a MYC polypeptide.

32. (canceled)

33. (canceled)

34. The composition of claim 1, wherein the HFSCR comprises at least four hair follicle stem cell reprogramming factors selected from the group consisting of: a nucleic acid encoding a p53 polypeptide, a nucleic acid encoding a NF-I polypeptide, a nucleic acid encoding a Lhx polypeptide, a nucleic acid encoding a TFAP2 polypeptide, a nucleic acid encoding an IRX polypeptide, and a nucleic acid encoding a Myc polypeptide.

35. (canceled)

36. (canceled)

37. (canceled)

38. The composition of claim 1, wherein the HFSCR comprises at least six hair follicle stem cell reprogramming factors comprising a nucleic acid encoding a p53 polypeptide, a nucleic acid encoding a NF-I polypeptide, a nucleic acid encoding a Lhx polypeptide, a nucleic acid encoding a TFAP2 polypeptide, a nucleic acid encoding an IRX polypeptide, and a nucleic acid encoding a Myc polypeptide.

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)


SEQUENCE LISTING (27 sequences)

SEQ ID NO: 1 - protein, 16 aa, Drosophila melanogaster
RQIKIWFQNRRMKWKK

SEQ ID NO: 2 - protein, 9 aa, Human immunodeficiency virus 1
RKKRRQRRR

SEQ ID NO: 3 - protein, 17 aa, Human immunodeficiency virus 1
TRQARRNRRRRWRERQR

SEQ ID NO: 4 - protein, 9 aa, synthetic construct
RRRRRRRRR

SEQ ID NO: 5 - protein, 8 aa, synthetic construct
RRRRRRRR

SEQ ID NO: 6 - DNA, 18 bp, synthetic construct
  source 1..18: mol_type = other DNA
ccaaccgcgagaagatga

SEQ ID NO: 7 - DNA, 20 bp, synthetic construct
  source 1..20: mol_type = other DNA
ccagaggcgtacagggatag

SEQ ID NO: 8 - DNA, 20 bp, synthetic construct
  source 1..20: mol_type = other DNA
ggccaggaaatcacatccta

SEQ ID NO: 9 - DNA, 20 bp, synthetic construct
  source 1..20: mol_type = other DNA
ggcagtgtctctccaaatcc

SEQ ID NO: 10 - DNA, 24 bp, synthetic construct
  source 1..24: mol_type = other DNA
agatgctgcttgacataaagacac

SEQ ID NO: 11 - DNA, 20 bp, synthetic construct
  source 1..20: mol_type = other DNA
gctaccaccacctcctgaag

SEQ ID NO: 12 - DNA, 24 bp, synthetic construct
  source 1..24: mol_type = other DNA
ggctttactcaagcagatctcatc

SEQ ID NO: 13 - DNA, 20 bp, synthetic construct
  source 1..20: mol_type = other DNA
cccttacccatgttgagacg

SEQ ID NO: 14 - DNA, 20 bp, synthetic construct
  source 1..20: mol_type = other DNA
ggacagcatgatgagcagaa

SEQ ID NO: 15 - DNA, 20 bp, synthetic construct
  source 1..20: mol_type = other DNA
caggcatggtatgtcctgaa

SEQ ID NO: 16 - DNA, 22 bp, synthetic construct
  source 1..22: mol_type = other DNA
aaacaccttgagccttaaaacg

SEQ ID NO: 17 - DNA, 20 bp, synthetic construct
  source 1..20: mol_type = other DNA
aggcaggaggaaatgcacta

SEQ ID NO: 18 - DNA, 20 bp, synthetic construct
  source 1..20: mol_type = other DNA
tgggaccacctggtattctg

SEQ ID NO: 19 - DNA, 22 bp, synthetic construct
  source 1..22: mol_type = other DNA
ccagcacaactcaatacaatgc

SEQ ID NO: 20 - DNA, 18 bp, synthetic construct
  source 1..18: mol_type = other DNA
agcctcttcggcttctcg

SEQ ID NO: 21 - DNA, 19 bp, synthetic construct
  source 1..19: mol_type = other DNA
ttggctctctgcagtggaa

SEQ ID NO: 22 - DNA, 19 bp, synthetic construct
  source 1..19: mol_type = other DNA
ctgggagagcaagtgttgg

SEQ ID NO: 23 - DNA, 22 bp, synthetic construct
  source 1..22: mol_type = other DNA
ggaacatacaaccagggaccta

SEQ ID NO: 24 - DNA, 22 bp, synthetic construct
  source 1..22: mol_type = other DNA
cccaaagaatgggtttgtattt

SEQ ID NO: 25 - DNA, 21 bp, synthetic construct
  source 1..21: mol_type = other DNA
tggaggtttcaagatcaccat

SEQ ID NO: 26 - DNA, 18 bp, synthetic construct
  source 1..18: mol_type = other DNA
gtgccggtggacgataag

SEQ ID NO: 27 - DNA, 18 bp, synthetic construct
  source 1..18: mol_type = other DNA
gagtccgggtggtagtgc